(19) United States
(12) Patent Application Publication
(10) Pub. No.: US 2026/0144908 A1
(43) Pub. Date: May 28, 2026

(54) METHOD AND APPARATUS FOR STERILIZING OBJECTS AND TRANSPORTING CONTAMINATED OBJECTS

(71) Applicant: Turbett Surgical, Inc., Henrietta, NY (US)

(72) Inventor: Robert E. Turbett, Penfield, NY (US)

(21) Appl. No.: 19/402,099

(22) Filed: Nov. 26, 2025

Related U.S. Application Data
(63) Continuation-in-part of application No. 19/361,510, filed on Oct. 17, 2025.
(60) Provisional application No. 63/726,111, filed on Nov. 27, 2024.
(60) Provisional application No. 63/895,526, filed on Oct. 8, 2025.

(51) Int. Cl. A61L 2/28; A61L 103/15

(52) U.S. Cl. CPC A61L 2/28; A61L 2103/15; A61L 2202/122

(57) ABSTRACT

An occlusion device for a vent port of a sterilizing container, the sterilizing container having an access port, a door connected to the sterilizing container, the door moveable between an open position permitting passage through the access port to an interior of the sterilizing container and a closed position precluding passage through the access port, at least one of the sterilizing container and the door having a vent port, the occlusion device comprising a nonporous sheet sized to encompass and occlude the vent port of the sterilizing container, the nonporous sheet comprising a material that is resistant to punctures or tears.

### CROSS-REFERENCE TO RELATED APPLICATIONS

The present application is a continuation in part of co-pending U.S. patent application Ser. No. 19/361,510 filed on Oct. 17, 2025 and incorporated herein by reference and claims the benefit of U.S. Provisional Ser. No. 63/726,111 filed Nov. 27, 2024, and U.S. Provisional Ser. No. 63/895,526 filed Oct. 8, 2025, the entire disclosure of each is hereby expressly incorporated by reference.

### FIELD OF THE INVENTION

Exemplary embodiments of the present invention relate to a method and apparatus for sterilizing and transporting objects that need to be sterilized, and more particularly to a method and apparatus for converting a sterilizing container into a container that be used to transport instruments needing to be sterilized.

### BACKGROUND OF THE INVENTION

Sterilization is a term referring to any process that eliminates (removes) or kills microbial life, including transmissible agents (such as fungi, bacteria, viruses, or spore forms) present on a surface, or contained in a fluid, or in medication, or in a compound such as biological culture media. Sterilization can be achieved by applying steam, heat, chemicals, including but not limited to ethylene oxide (EtO) sterilization, gamma irradiation, electron beam (e-beam), high pressure, hydrogen peroxide vaporization, dry heat, and filtration or combinations thereof.

In general, surgical instruments, implants, medications, and other objects that enter an already aseptic part of the body (such as the bloodstream, or penetrating the skin) must be sterilized to a high sterility assurance level. Examples of such instruments include scalpels, hypodermic needles, endocavity probes, endoscopes, and implantable medical devices (IMD), such as artificial pacemakers and implantable joint devices.

A widely used method for steam sterilization is the autoclave. Autoclaves commonly use steam heated to 121-134° C. To achieve a degree of sterility, a holding time of at least 15 minutes at 121° C. at 100 kPA, or 3 minutes at 134° C. at 100 kPa is typically required. Additional sterilizing time is usually required for liquids and instruments packed in layers of cloth, as they may take longer to reach the required temperature.

One method of sterilization involves passing steam through a cabinet or container having at least one vented area. In some configurations, the vented area comprises at least one fenestrated area or at least one vent port. The vented area allows steam to penetrate the cabinet or container during the sterilization process. For example, U.S. Pat. Nos. 9,616,368 and 9,724,438 of Turbett et al., which is hereby incorporated by reference, describes a sterilizing cabinet assembly. The sterilizing cabinet includes a cabinet having an access port and a door connected to the cabinet, with at least one of the cabinet and the door including a vent port. During the sterilization process, typically at least one filter overlies the vented area. The filter or filters allow the sterilizing agent, for example, high temperature steam or chemical agents, to pass through the cabinet or container while preventing bacteria, viruses, particles, and other extraneous materials from entering sterilizing cabinet or container before, during, or after the sterilizing process. Other sterilization systems use a mechanical filtering system comprising at least one valve that has an open position and a closed position such that the valve is in the open position during the sterilization cycle and the closed position post-sterilization cycle. For effective sterilization, steam needs to penetrate the cabinet or container load uniformly. Accordingly, the cabinet or container must not be overcrowded, and the lids of bottles must be left ajar. During the initial heating of the chamber, residual air must be removed. Indicators should be placed in the most difficult places for the steam to reach to ensure that steam actually penetrates there.

Once the sterilizing process is completed the filter needs to be removed and inspected by medical professionals to verify the integrity of the sterilizing process was maintained. If it is discovered during inspection that the filter did not remain intact, the sterilizing process has to be repeated with a new filter.

After sterilization, the instruments are transported to the place or area of use. Typically, the area of use is an operating room or patient room where a medical procedure is to be performed. Following the procedure, the now contaminated instruments, medical devices, objects, wrappers, drapes, gloves, and other contaminated materials used during the procedure must be transported to the area where the instruments, devices and contaminated materials can be property discarded or decontaminated and reprocessed. For example, hospitals and outpatient facilities typically have a Sterile Processing Department (SPD) where surgical and procedural instruments and medical devices are reprocessed and sterilized. Alternatively, the items can be brought to a facility off-site to be decontaminated and reprocessed.

Trays, baskets, sterilizing cabinets, and sterilizing containers cannot be used to transport the contaminated instruments as rules and regulations require contaminated instruments to be transported in a closed container or an enclosed cart since the contaminations can carry pathogens harmful to others. Conventionally, the closed container must be leak-proof and puncture resistant. Such containers are typically labeled with a biohazard symbol such that it is easily identified biohazardous. As sterilizing containers and cabinets have vent ports and filters subject to puncture, such devices do not meet these requirements and are not appropriate for the purpose of transporting soiled instruments and other objects to the reprocessing area. Conventionally, the soiled instruments and other objects are typically placed in a secondary container and/or bag for transport. These secondary containers must be stored when not in use, taking up storage space. It requires time to acquire the appropriate number of secondary containers, and not having enough for all the soiled instruments post-procedure can be problematic. Further, secondary containers add to the cost of equipment needed. Also, having to use secondary containers creates an additional step in preparing and cleaning post-procedure, which already involves many steps of gathering instruments, disinfection and waste management. For example, after contaminated objects are transported in the secondary containers, the secondary containers must be washed before re-use. Where bags and other disposable materials are used as secondary containers, extra waste is generated.

Therefore, the need exists for improved systems and methods for sterilizing objects and then transporting the contaminated objects to be processed and sterilized.

### SUMMARY OF THE INVENTION

In view of the foregoing, it is an object of the present invention to provide a method and apparatus for sterilization and post-procedure transportation of contaminated instruments, devices and materials.

Generally, the present disclosure provides a sterilizing container assembly having both a sterilization configuration and a transportation configuration, comprising a container having an access port, a door connected to the container, the door moveable between an open position permitting passage through the access port to an interior of the container and a closed position precluding passage through the access port, and at least one of the container and the door having a vent port, wherein a filter overlies the vent port in the sterilization configuration and forms a sealed interface with an adjacent portion of the at least one of the container and the door and wherein an occlusion device overlies the vent port in the transportation configuration and forms a sealed interface with an adjacent portion of the at least one of the container and the door.

The present disclosure contemplates that the gasket can comprise an edge portion having a thickness sufficient to form a sealed interface between at least one of the container and the door to prevent extraneous materials from exiting the container. The gasket can be permanently affixed or integral with the nonporous cover or the gasket can be separable from the nonporous cover. The gasket can be coextensive with the nonporous cover.

The nonporous cover can include an edge portion having a thickness sufficient to form a sealed interface between at least one of the container and the door to prevent extraneous materials from exiting the container.

The sterilizing container assembly can comprise a frame portion defining the vent port, wherein the nonporous cover is an insert having an inner portion for overlying the vent port and an outer edge portion for overlying the frame portion, the outer edge portion forming the sealed interface with at the adjacent portion of the at least one of the container and the door.

The vent port can be located on the container. The first filter can occlude the vent port when the sterilizing container is in the sterilization configuration and the nonporous cover occludes the vent port when the sterilizing container is in the transportation configuration. The door can be removeable and replaced with the nonporous cover in the transportation configuration.

In one configuration, the present disclosure provides a sterilizing container assembly having an enclosing wall including sidewalls, a top wall and a bottom wall, wherein the access port is lying in a plane spaced from the rear wall of the enclosing wall. The access port can be sized to pass a plurality of trays, each tray sized to retain a plurality of objects.

Alternatively, the sterilizing container assembly can comprise an enclosing wall comprising a bottom wall and sidewalls, wherein the access port is lying in a plane spaced from the bottom wall of the enclosing wall.

The present disclosure provides, in one configuration, that the nonporous cover is sterilizable. The nonporous cover can be fastened to the container to form the sealed interface between the nonporous cover and the adjacent portion of at least one of the container and the door. The fastener can comprise clamps hingedly affixed to the container. The fastener can comprise clamps separable from the container. The nonporous cover can be resistant to punctures or tears and can include a biohazard indicator. The biohazard indicator can be at least one of a color or a label.

In another configuration, the present disclosure provides a sterilizing container assembly for use in a sterilization process and a transportation process, the sterilizing container assembly comprising a container having an access port for passing an object or tray, a first separable door for coupling to the container, the first separable door moveable between an open position permitting passage through the access port to an interior of the container and a closed position precluding passage through the access port, the first separable door having a vent port; and a nonporous cover sized to overlie the vent port to prevent contaminants from exiting the container. The nonporous cover can be configured to form a seal with the door. The sterilizing container assembly can include a screen sized to overlie the nonporous cover, the screen having a vent port that is occluded by the nonporous cover during the transportation process.

In yet another configuration, the present disclosure includes a method for using a sterilizing container as a transportation container for contaminated objects, the method comprising disposing at least one contaminated object into a sterilizing container having at least one vent port, overlying a nonporous cover over the vent port to occlude the vent port of the container, and securing the nonporous cover to the sterilizing container. The step of securing the nonporous cover can comprise the step of engaging at least one clamp on the sterilizing container to form a sealed interface between the nonporous cover and the sterilizing container. The step of securing the nonporous cover can comprise the step of applying at least one clamp to secure the nonporous cover to the sterilizing container.

In another configuration, the present disclosure includes a method for using a sterilizing container as a transportation container for contaminated objects, the method comprising disposing at least one contaminated object into a sterilizing container having an access port, at least one vent port, and a door connected to the sterilizing container, the door moveable between an open position and a closed position precluding passage through the access port, overlying a nonporous cover over the vent port to occlude the vent port of the container, and securing the nonporous cover to the sterilizing container to form a sealed interface with an adjacent portion of one of the sterilizing container and the door. The method can further comprise the step of securing the door to the sterilizing container. The nonporous cover and the door can be secured to the sterilizing container to occlude the access port and vent port.

The present disclosure in a further configuration includes a method for using a sterilizing container as a transportation container for contaminated objects, the method comprising disposing at least one contaminated object into a sterilizing container having an access port, at least one vent port, and a door connected to the sterilizing container, the door moveable between an open position and a closed position precluding passage through the access port, overlying a nonporous cover over the vent port to occlude the vent port of the container, and securing the nonporous cover to the sterilizing container or the door to form a sealed interface with an adjacent portion of one of the sterilizing container and the door. The door can be removable from the sterilizing container and comprises the vent port.

Another configuration of the present disclosure includes an occlusion device for a vent port of a sterilizing container, the occlusion device comprising a nonporous sheet sized to occlude a vent port of a sterilizing container, and a gasket comprising an edge portion having a thickness sufficient to form a sealed interface between at least one of the sterilizing container and the door to prevent extraneous materials from exiting the container. The occlusion device can include a gasket that is either permanently affixed or integral with the nonporous cover, separable from the nonporous cover, or coextensive with the nonporous cover. A nonporous sheet can be disposed between the door having a vent port and the sterilizing container to occlude the vent port. Alternatively, the nonporous sheet is disposed between an enclosing wall of the sterilizing container and a screen to occlude the vent port.

Yet another configuration of the present disclosure includes an occlusion device for a vent port of a sterilizing container, the sterilizing container having an access port, a door connected to the sterilizing container, the door moveable between an open position permitting passage through the access port to an interior of the sterilizing container and a closed position precluding passage through the access port, at least one of the sterilizing container and the door having a vent port, the occlusion device comprising a nonporous sheet sized to encompass and occlude the vent port of the sterilizing container, the nonporous sheet comprising a material that is resistant to punctures or tears.

A configuration of the present disclosure includes a method for using a sterilizing container as a transportation container for contaminated objects, the method comprising sterilizing at least one object in a sterilizing container having an access port to allow access to an interior of the sterilizing container, a door connected to the sterilizing container, the door moveable between an open position and a closed position precluding passage through the access port, at least one vent area on the door or the sterilizing container, the vent area having at least one vent port, and at least one filter overlying the vent port, moving the door from the closed position to the open position to allow access to the interior of the sterilizing container, removing the at least one filter overlying the vent port, removing the at least one object in the sterilizing container, overlying an occlusion device over the vent port area to occlude the vent port, securing to the vent area the occlusion device within an interior of the sterilizing container or the door to encompass and occlude the vent port and to form a sealed interface to preclude contaminants from exiting the container through the vent port, disposing at least one contaminated object into the sterilizing container, and moving the door to the closed position to preclude passage through the access port.

Another configuration of the present disclosure includes a method for using a sterilizing container as a transportation container for contaminated objects, the method comprising sterilizing at least one object in a sterilizing container having an access port to allow access to an interior of the sterilizing container, a door connected to the sterilizing container, the door moveable between an open position and a closed position precluding passage through the access port, at least one vent area on the door or the sterilizing container, the vent area having at least one vent port, and at least one filter overlying the vent port, sterilizing at least one object in a sterilizing container having an access port to allow access to an interior of the sterilizing container, a door connected to the sterilizing container, the door moveable between an open position and a closed position precluding passage through the access port, at least one vent area on the door or the sterilizing container, the vent area having at least one vent port, and at least one filter overlying the vent port, moving the door from the closed position to the open position to allow access to the interior of the sterilizing container, removing the at least one filter overlying the vent port on either the door or the sterilizing container, removing the at least one object in the sterilizing container, encasing at least one contaminated object in a nonporous material comprising a flexible film that is resistant to punctures or tears to preclude contaminants from exiting the container, disposing the encased contaminated object into the sterilizing container, closing the door to the sterilizing container, and transporting the sterilizing container having the encased contaminated object to a processing area.

The following will describe embodiments of the present invention, but it should be appreciated that the present invention is not limited to the described embodiments and various modifications of the invention are possible without departing from the basic principle. The scope of the present invention is therefore to be determined solely by the appended claims.

### BRIEF DESCRIPTION OF THE SEVERAL VIEWS OF THE DRAWING(S)

FIG. 1 is a front view of a configuration of a sterilizing container suitable for use in practicing exemplary embodiments of this invention.

FIG. 2 is a perspective view of the sterilizing container shown in FIG. 1 suitable for use in practicing exemplary embodiments of this invention.

FIG. 3A is side view of a filter arrangement of a sterilizing container for use in practicing exemplary embodiments of this invention.

FIG. 3B is a side view of an alternative configuration of a filter arrangement of a sterilizing container for use in practicing exemplary embodiments of this invention.

FIG. 3C is a side view of an occlusion device arrangement of a sterilizing container for use in practicing exemplary embodiments of this invention.

FIG. 3D side view of an occlusion device arrangement of a sterilizing container for use in practicing exemplary embodiments of this invention.

FIG. 4A is a perspective view of an alternative configuration of a sterilizing container suitable for use in practicing exemplary embodiments of this invention.

FIG. 4B is a top view of bottom section of a filter arrangement of a sterilizing container suitable for use in practicing exemplary embodiments of this invention.

FIG. 4C is a top view of a middle section of a filter arrangement of a sterilizing container suitable for use in practicing exemplary embodiments of this invention.

FIG. 4D is a top view of top section of a filter arrangement of a sterilizing container suitable for use in practicing exemplary embodiments of this invention.

FIG. 4E is a top view of bottom section of an occlusion device arrangement of a sterilizing container suitable for use in practicing exemplary embodiments of this invention.

FIG. 4F is a top view of a middle section of an occlusion device arrangement of a sterilizing container suitable for use in practicing exemplary embodiments of this invention.

FIG. 4G is a top view of top section of an occlusion device arrangement of a sterilizing container suitable for use in practicing exemplary embodiments of this invention.

FIG. 5A is a perspective view of an alternative sterilizing container suitable for use in practicing exemplary embodiments of this invention.

FIG. 5B is a front view of an alternative filter arrangement of a sterilizing container suitable for use in practicing exemplary embodiments of this invention.

FIG. 5C is a front view of the top portion of an alternative filter arrangement of a sterilizing container suitable for use in practicing exemplary embodiments of this invention.

FIG. 5D is a front view of an alternative occlusion device arrangement of a sterilizing container suitable for use in practicing exemplary embodiments of this invention.

FIG. 5E is a front view of the top portion of an alternative occlusion device arrangement of a sterilizing container suitable for use in practicing exemplary embodiments of this invention.

FIG. 5F is front view of an alternative occlusion device arrangement of a sterilizing container suitable for use in practicing exemplary embodiments of this invention.

FIG. 5G is a front view of the top portion of an alternative occlusion device arrangement of a sterilizing container suitable for use in practicing exemplary embodiments of this invention.

FIG. 6A is a perspective of an alternative filter arrangement of a sterilizing container suitable for use in practicing exemplary embodiments of this invention.

FIG. 6B is a perspective of an alternative filter arrangement of a sterilizing container suitable for use in practicing exemplary embodiments of this invention.

FIG. 6C is a perspective of an alternative occlusion device arrangement of a sterilizing container suitable for use in practicing exemplary embodiments of this invention.

FIG. 6D is a perspective of an alternative occlusion device arrangement of a sterilizing container suitable for use in practicing exemplary embodiments of this invention.

FIG. 6E is a perspective of an alternative occlusion device arrangement of a sterilizing container suitable for use in practicing exemplary embodiments of this invention.

FIG. 6F is a perspective of an alternative occlusion device arrangement of a sterilizing container suitable for use in practicing exemplary embodiments of this invention.

FIG. 7 is a perspective view of an exemplary sterilizing container having a filter door suitable for use in practicing exemplary embodiments of the present disclosure.

FIG. 8 is a perspective view of an alternative embodiment of a sterilizing container suitable for use in practicing exemplary embodiments of this disclosure.

FIG. 9 is a perspective view of a door suitable for use in practicing exemplary embodiments of this invention.

FIG. 10 is a magnified cross-sectional view of sterilizing container and a filter door suitable for use in practicing exemplary embodiments of this disclosure.

FIG. 11 is a perspective view of an exemplary sterilizing container and filter door suitable for use in practicing exemplary embodiments of the present disclosure.

FIG. 12 is a perspective exploded view of an exemplary sterilizing container showing a transportation configuration suitable for use in practicing exemplary embodiments of the present disclosure.

FIG. 13 is a perspective exploded view of an alternative sterilizing container showing a transportation configuration suitable for use in practicing exemplary embodiments of the present disclosure.

FIG. 14 is a perspective exploded view of an alternative sterilizing container showing a transportation configuration suitable for use in practicing exemplary embodiments of the present disclosure.

FIG. 15 is a perspective exploded view of an alternative sterilizing container showing a transportation configuration suitable for use in practicing exemplary embodiments of the present disclosure.

FIG. 16 is a perspective exploded view of an alternative sterilizing container showing a transportation configuration suitable for use in practicing exemplary embodiments of the present disclosure.

FIG. 17A is a perspective view of an occlusion device suitable for use in practicing exemplary embodiments of this invention.

FIG. 17B is a side view of an occlusion device suitable for use in practicing exemplary embodiments of this invention.

FIG. 18A is a perspective view of an alternative occlusion device suitable for use in practicing exemplary embodiments of this invention.

FIG. 18B is a side view of an alternative occlusion device suitable for use in practicing exemplary embodiments of this invention.

FIG. 19A is a perspective view of another occlusion device suitable for use in practicing exemplary embodiments of this invention.

FIG. 19B is a side view of another occlusion device suitable for use in practicing exemplary embodiments of this invention.

FIG. 20 is a perspective view of an occlusion device cartridge suitable for use in practicing exemplary embodiments of this invention.

FIG. 21 is a perspective view of an alternative occlusion device cartridge suitable in practicing exemplary embodiments of this invention.

FIG. 22 is a perspective view of an alternative occlusion device cartridge suitable in practicing exemplary embodiments of this invention.

FIG. 23 is a perspective view of another occlusion device cartridge suitable in practicing exemplary embodiments of this invention.

FIG. 24 is a logic diagram of a method of using the sterilizing container having a transport configuration for contaminated objects and suitable for use in practicing exemplary embodiments of this disclosure.

FIG. 25 is a perspective exploded view of an alternative sterilizing container showing a transportation configuration suitable for use in practicing exemplary embodiments of the present disclosure.

FIG. 26 is a perspective exploded view of an alternative sterilizing container showing yet another transportation configuration suitable for use in practicing exemplary embodiments of the present disclosure.

FIG. 27A is a cross-sectional view of a nonporous sheet having an adhesive layer.

FIG. 27B is a cross-sectional view of a nonporous sheet having an adhesive layer along the perimeter edge.

FIG. 28 is a perspective exploded view of an alternative sterilizing container showing another transportation configuration suitable for use in practicing exemplary embodiments of the present disclosure.

FIG. 29 is a perspective exploded view of an alternative sterilizing container showing yet another transportation configuration suitable for use in practicing exemplary embodiments of the present disclosure.

FIG. 30 is a logic diagram of a method of using the sterilizing container having a transport configuration for contaminated objects and suitable for use in practicing exemplary embodiments of this disclosure.

FIG. 31 is a logic diagram of a method of using the sterilizing container having a transport configuration for contaminated objects and suitable for use in practicing exemplary embodiments of this disclosure.

### DETAILED DESCRIPTION OF THE INVENTION

In the medical field, it is of the utmost importance that medical instruments are sterilized prior to any medical procedure. This drastically helps prevent the spread of infectious materials. In the marketplace, there are a wide variety of devices that provide for sterilization of medical instruments through the use of a sterilizing agent, such as steam. Instrument trays can be wrapped in a cloth or paper that acts as a filter, allowing the tray to be sterilized, then delivered to the operating room. Alternatively, a rigid container can contain the instrument tray. Typically, the device (e.g., a rigid container) contains a vent for venting the steam used to sterilize the contents of the device. A disposable filter usually covers these vents. The filters have two major purposes. First, they prevent extraneous materials from entering the sterilizing device during and after the sterilization cycle. Second, they allow sterilizing steam to enter and exit the sterilizing device.

In order to check that the integrity of the sterilization cycle has been maintained a person (usually a medical technician) must verify that there are no holes or other types of rips in the filter. This is done by removing the filter from the sterilizing device and visually inspecting the filter. If there are no holes or other types of punctures, or rips in the filter, the instruments are transported to the place or area of use. Typically, the area of use is an operating room or patient room where a medical procedure is to be performed.

Following the procedure, the instruments, medical devices, and other contaminated materials used during the procedure must be transported from the clinical area or office location to another location where the instruments, devices and contaminated materials can be decontaminated and reprocessed to be re-used in another procedure. Typically, the contaminated instruments are rinsed after surgery and/or presoaked. Additionally, it can be necessary to manually scrub the instruments or utilize mechanical cleaning devices such as ultrasonic cleaners or washer-disinfectors. Next, the instruments are sterilized. Hospitals and outpatient facilities typically have a Sterile Processing Department (SPD) where surgical and procedural instruments and medical devices are reprocessed and sterilized. Alternatively, the contaminated objects are brought offsite to a central processing center.

Trays, baskets, sterilizing cabinets, and other types of sterilizing containers cannot be used to transport the contaminated objects, such as instruments used in a medical procedure, as rules and regulations require contaminated objects to be transported in a closed container or an enclosed cart since the contaminations can carry pathogens harmful to others. In some applications, the closed container must be leak proof and puncture resistant. As sterilizing containers and cabinets have vent ports and filters subject to puncture, such devices do not meet these requirements and are not appropriate for the purpose of transporting soiled instruments to the reprocessing area. Thus, the soiled instruments are typically placed in a secondary container for transport. These secondary containers are made of metal or plastic and have tight-fitting lids to prevent spilling in the event the container is dropped or jostled. Sometimes the containers are further wrapped in bags which are then disposed of after use creating additional waste. An object of exemplary embodiments of this disclosure provide a solution to this problem.

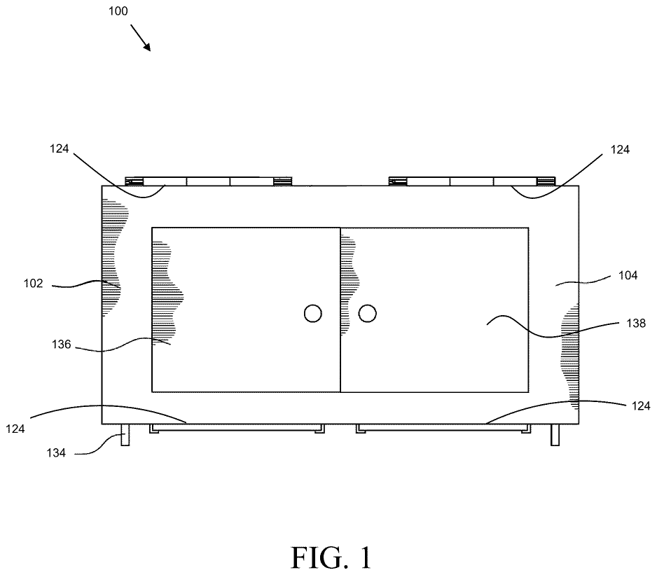

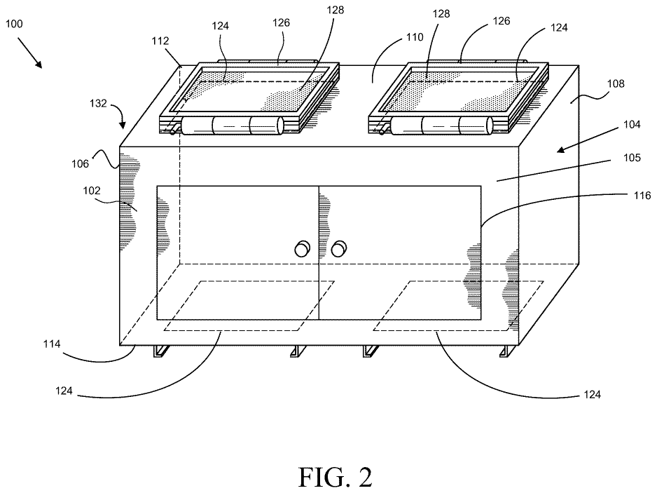

Referring to FIGS. 1-3B, is a perspective view a sterilizing container 100. As shown in FIG. 1, the sterilizing container 100 can be a sterilizing cabinet 102 having an enclosing wall 104 including sidewalls 106, 108, top wall 110, rear wall 112 and bottom wall 114 forming a sterilizing cabinet frame 132. In some configurations, the sterilizing cabinet 102 enclosing wall 104 includes a front wall 105 or a portion thereof. An access port 116 can be lying in a plane and spaced from the rear wall 112. The access port 116 is sized to pass a plurality of trays (not shown), each tray sized to retain a plurality of objects (not shown). In this configuration, sterilizing container 100 further includes vents 124, filter holder 126, and legs 134. Door 136 or doors 136, 138 are able to open and close for access to the interior of sterilizing cabinet 102. In this configuration, door 136 or doors 136, 138 are physically connected to sterilizing cabinet frame 132. Door 136 or doors 136, 138 can be attached through the use of a hinge or hinges (not shown) which allows the doors 136 or doors 136, 138 to swing open. Alternatively, door 136 or doors 136, 138 can be removable from sterilizing cabinet 102 through the use of clamps (not shown in FIG. 1) or through the use of other fasteners. It should be appreciated that exemplary embodiments of door 136 or doors 136, 138 include any mechanism that allows for door 136 or doors 136, 138 to move from an open position to a closed position to provide access to the interior of sterilizing cabinet 102. The door 136 or doors 136, 138 form a sealed interface between the door 136 or doors 136, 138 and the sterilizing cabinet 102. Sterilizing cabinet 102 in this embodiment provides for four vents 124. However, it should be appreciated that exemplary embodiments of sterilizing cabinet 102 are not limited to four vents. Exemplary embodiments of sterilizing cabinet 102 can include one or more vents 124. Two vents 124 in the top wall 110 of sterilizing cabinet 102 and two vents 124 in the bottom wall 114 of sterilizing cabinet 102 are shown in FIGS. 1 and 2. Vents 124 provide numerous small openings for the passage of sterilizing steam. The small openings in vents 124 can be holes or slits. Alternatively, vents 124 can be fenestrated. A fenestrated vent surface with numerous openings allows for the passage of a sterilizing agent, such as steam from an autoclave during a sterilization cycle. In the sterilization configuration, filters, for example filter 128 and/or secondary filter 130, overlie the vents 124. In the transportation configuration, occlusion device 200 overlies the vents 124 as described in more detail below.

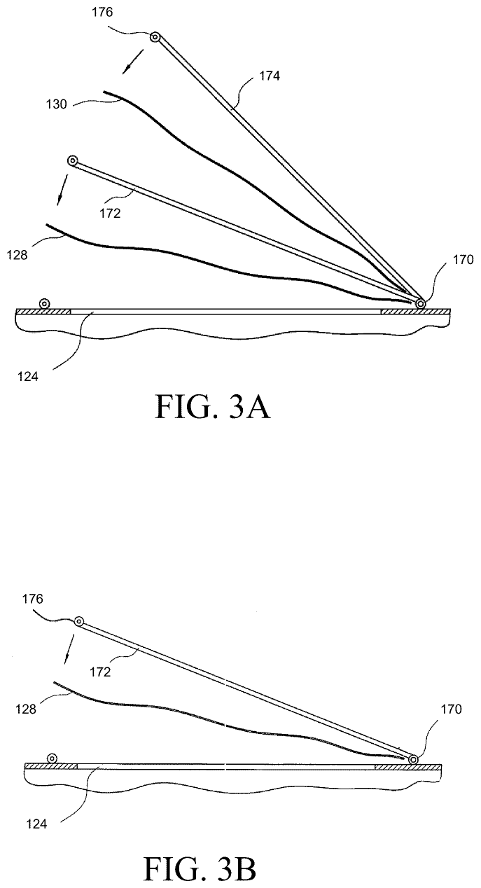

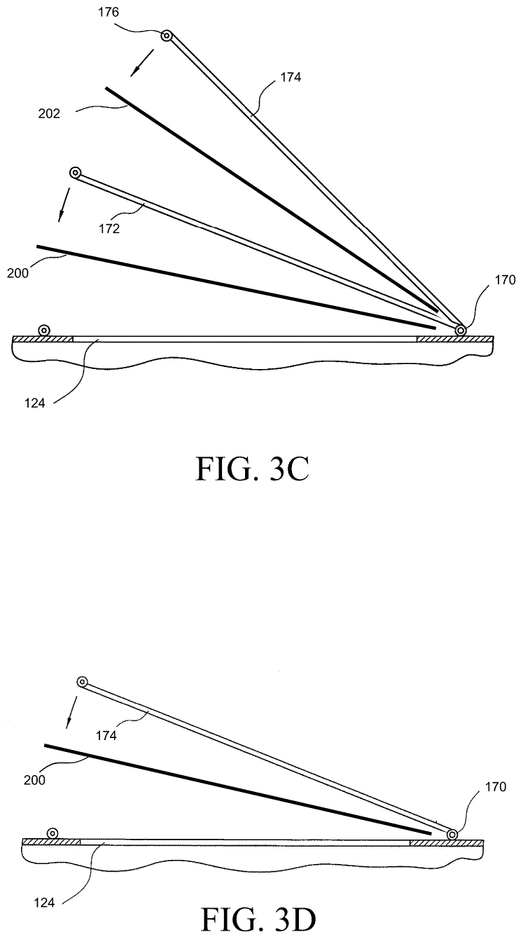

In the sterilization configuration, as shown in FIGS. 2-3B , primary filter 128 in conjunction with filter holder 126 covers vent 124. Primary filter 128, with filter holder 126, forms a seal with the adjacent portions of sterilizing cabinet 100 such that during the operation of a sterilizing cycle, any sterilizing steam that passes through the vent 124 must then pass through primary filter 128. Primary filter 128 can be made of a very thin paper. Exemplary embodiments provide that primary filter 128 can be made of any porous material that (1) allows for the passage of sterilizing steam from sterilizing cabinet 102 and (2) prevents extraneous materials from passing through primary filter 128 and entering vent 124. Primary filter 128 is removable from sterilizing cabinet 102 and is typically replaced with a new filter following each sterilizing cycle.

Secondary filter 130 resides on top of primary filter 128 in filter holder 126. Secondary filter 130 covers primary filter 128 and forms a seal with primary filter 128 through filter holder 126 such that any sterilizing steam that passes through the vent 124 must then pass through primary filter 128 and secondary filter 130. Secondary filter 130 can be made of a very thin paper. Secondary filter 130 can be made of any porous material that (1) allows for the passage of sterilizing steam from sterilizing cabinet 102 and primary filter 128 and (2) prevents extraneous materials from passing through secondary filter 130. The primary filter 128 and/or secondary filter 130 can comprise a filter cartridge. The filter cartridge can comprise a single filter or multiple filters. A seal or gasket can be included between multiple filters or on the outside of at least one of the filters.

Exemplary embodiments of this disclosure provide for secondary filter 130 to form a sealed periphery with primary filter 128. In another exemplary embodiment the sealed interface between the primary filter 128 and the adjacent portion of either the sterilizing cabinet 102 is independent of an interface between secondary filter 130 and primary filter 128. One exemplary arrangement provides for primary filter 128 and secondary filter 130 to be coextensive. In another exemplary embodiment primary filter 128 and secondary filter 130 have different filter properties. For instance, primary filter 128 and secondary filter 130 can filter different elements of the sterilizing agent which exits sterilizing cabinet 102 during a sterilization cycle. In an alternative exemplary embodiment primary filter 128 and secondary filter 130 have similar filter properties. Another exemplary embodiment provides that primary filter 128 and secondary filter 130 are different colors.

Exemplary embodiments of this arrangement provide for a seal between filter holder 126 and primary filter 128, between filter holder 126 and secondary filter 130 and sterilizing cabinet 102. This seal serves two primary purposes. First, it forces all of the sterilizing agent that enters and exits the sterilizing cabinet 102 to pass through the filters. Second, it keeps extraneous materials from entering the sterilizing cabinet 102 through vents 124, which are covered by primary filter 128 and secondary filter 130. It can be appreciated that exemplary embodiments of the sealed interface between the primary filter 128 and the sterilizing cabinet 102 includes both direct contact between primary filter 128 and sterilizing cabinet 102 as well as indirect contact between primary filter 128 and sterilizing cabinet 102 through the use of a sealing agent, such as caulk or an adhesive. In one exemplary embodiment, the seal is a silicone seal. Likewise, the sealed interface between the secondary filter 130 and the primary filter 128 or filter holder 126 includes both direct contact between primary filter 128 or filter holder 126 as well as indirect contact through the use of a sealing agent, such as caulk or an adhesive.

In yet another exemplary embodiment, primary filter 128 can be the only filter that covers vent 124. Here, primary filter 128 is removably held or maintained in place over vent 124 by filter holder 126. In this embodiment, there is no secondary filter. Primary filter 128 forms a seal with the adjacent portions of sterilizing cabinet 102 such that during the operation of a sterilizing cycle, any sterilizing steam that passes through the vent 124 must then pass through primary filter 128. Again, in this embodiment, primary filter 128 can be made of any porous material that (1) allows for the passage of sterilizing steam from sterilizing cabinet 100 and (2) prevents extraneous materials from passing through primary filter 128 and entering vent 124. Primary filter 128 is removable from sterilizing cabinet 102 and is typically replaced with a new filter following each sterilizing cycle. The filter holder 126 can include a swinging hinge which allows for the individual attachment and release of primary filters 128 and secondary filters 130. In alternative embodiments, the filter holders 126 do not include a hinge which allows for the independent removing of primary filters 128 and secondary filters 142. In this exemplary embodiment filter holders 126 allow for independently removing primary filter 128 and secondary filter 130 through the use of a sliding mechanism. Secondary filter 130 can be removed by sliding it out of filter holder 126 while maintaining primary filter's 128 seal with sterilizing cabinet 102 around vent 124.

In practice, an exemplary embodiment of a process of placing primary filter 128 and secondary filter 130 includes disposing primary filter 128 to overlie a vent 124 of sterilizing cabinet 102 and forming a first sealed interface with the sterilizing cabinet 102. The process continues by forming a second sealed interface between secondary filter 130 (or confirmatory filter) and at least a portion of one of sterilizing cabinet 102 and primary filter 128, at least a portion of the secondary filter 130 overlying a portion of the primary filter 128. The process can continue by passing a sterilizing agent (typically steam) through primary filter 128 and secondary filter 130 and vent 124.

Also in practice, an exemplary embodiment of a process of placing only a primary filter 128 includes disposing primary filter 128 with filter holder 126 to occlude vent 124 of sterilizing cabinet 102 creating a sealed interface with the sterilizing cabinet 102. The process can continue by passing a sterilizing agent (typically steam) through primary filter 128 and vent 124. The process can then conclude with verifying the integrity of primary filter 128 by either inspecting primary filter 128 while it covers vent 124 in filter holder 126 or after it is removed from filter holder 126. The process can be repeated if it is determined that the integrity of primary filter 108 was compromised during the sterilizing process.

It should be noted that exemplary embodiments of a sterilizing agent include any substance that provides for the destruction or elimination of living organisms, which often include heat, steam, pressure, gas, plasma, irradiation, chemical compounds, and chemical vapor. In exemplary embodiments sterilizing cabinet 102 can include a steam exposure indicator on either the primary filter 128 or the secondary filter 130 which designates when steam from sterilizing cabinet 102 has passed through one of the filters. An example of one such steam exposure indicator is a tape that changes colors when exposed to steam.

Referring to FIG. 3A, provided is a side view of the different elements and the type of movement allowed for filter holder 126 in exemplary embodiments of this invention. The sterilization configuration includes sterilizing cabinet 102, vent 124, primary filter 128, secondary filter 130, hinge 170, filter holder 126 section 172 which secures primary filter 128, and filter holder 126 section 174 which secures secondary filter 130. In this exemplary embodiment sections 172 and 174 are able to rotate about hinge 170 and can be moved from the closed position (covering vent 124) to the open position (not covering vent 124) independent of each other. For instance section 174 can be moved to the open position while section 172 remains in the closed position. Filter holder 126 can be temporarily secured by locking mechanism 176, such as a pin engaging an opening, clamp or latch. It can be appreciated that exemplary embodiments of these teachings provide for any mechanism that allows sections 172 and 174 of filter holder 126 to be maintained securely in the closed position and then opened when desired.

FIG. 3B provides a perspective view of the movement of an alternative filter arrangement of a sterilizing cabinet for use as a sterilizing container in practicing exemplary embodiments of this invention. Shown in FIG. 3B is the outside face of sterilizing cabinet 102, vent 124, primary filter 128, hinge 170, and filter holder 126 section 172, which secures primary filter 128. It should be noted that in this exemplary embodiment, there is only one filter (i.e., primary filter 128) and one filter holder 126 section 172. Here, filter holder 126 section 172 is able to rotate about hinge 170. It can be moved from the closed position (covering vent 124) to the open position (not covering vent 124. Filter holder 126 can be temporarily secured by locking mechanism 176, such as a pin engaging an opening, clamp or latch. It can be appreciated that exemplary embodiments of these teachings provide for any mechanism that allows sections 172 and 174 of filter holder 126 to be maintained securely in the closed position and then opened when desired.

The transportation configuration of the exemplary embodiment shown in FIGS. 1 and 2 includes the sterilizing cabinet 102, vent 124, and hinge 170, filter holder 126 section 172, and filter holder 126 section 174. Instead of primary filter 128 and/or secondary filter 130 overlying the vent 124, an occlusion device 200 overlies the vent 124. For example, as shown in FIGS. 3C and 3D, a nonporous occlusion device 200 overlies the vent port or vent ports 124 in place of the primary filter 128 to occlude the vent port or vent ports 124. In one configuration, filter holder 126 section 172 secures the occlusion device 200. In another configuration, filter holder 126 section 174 secures a secondary occlusion device 202 as shown in FIG. 3C. In another configuration, filter holder 126 section 174 does not hold an occlusion device such that only one occlusion device 200 is utilized in the transportation configuration as shown in FIG. 3D.

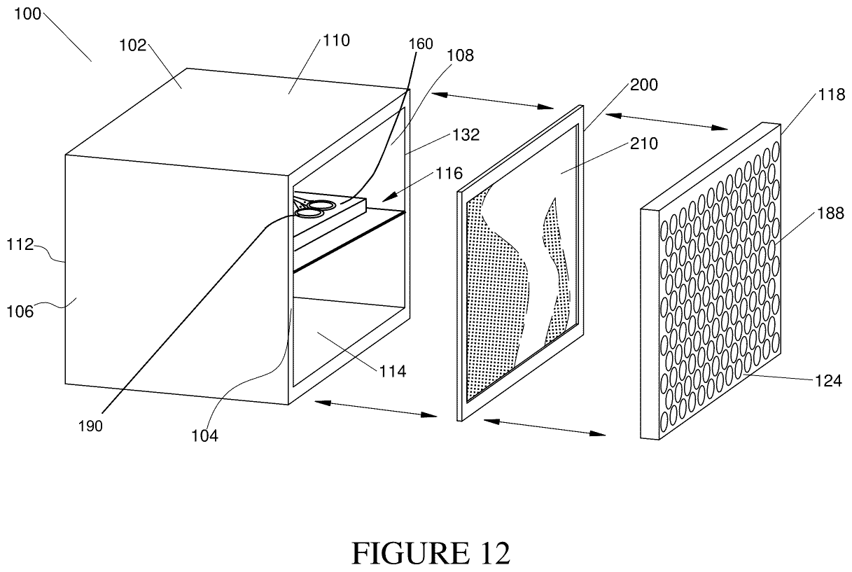

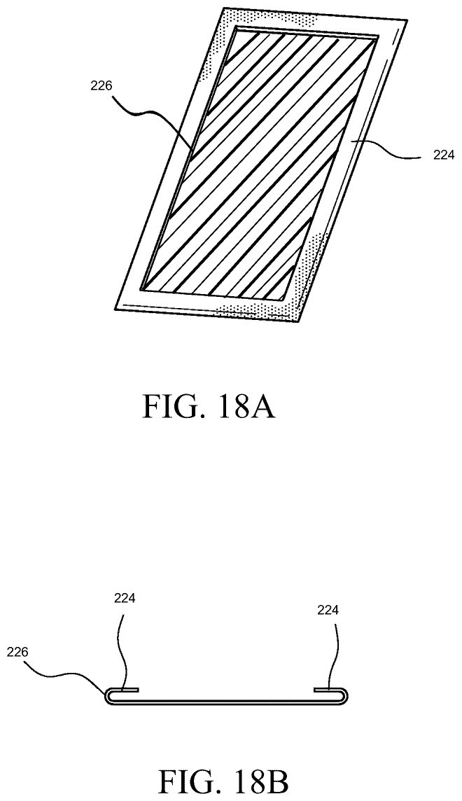

That is, a second nonporous occlusion device 202 can overlie the vent port or vent ports 124 in place of filter 130 to occlude the vent port or vent ports 124. In one configuration, the occlusion device 200, 202 is a sheet comprising a nonporous material. In another configuration, the occlusion device 200, 202 is a cover having a thickness and rigidity for occluding the vent ports 124. By rigidity it is meant that the occlusion device is capable of holding its shape without further support. The occlusion device 200 in one configuration forms a sealed interface with an adjacent portion of the at least one of the cabinet 102 and the filter holder 126 such that during the transportation of the cabinet 102 having at least one object 190, which can include at least one tray 160 (for example as shown in FIG. 12), the cabinet 102 is sealed to provide a leak-resistant transport system that contains contamination within the cabinet 102. In addition to being leak-resistant, the occlusion device 200 is tear and puncture resistant, thereby reducing the risk of injury and further containing the contaminated object(s) 190. In an exemplary embodiment, the occlusion device 200 is single-use and disposable after one use. In another exemplary embodiment, the occlusion device 200 is re-usable and washable along with the contaminated instruments. In certain exemplary embodiments, the occlusion device 200 is sterilizable. In a configuration, the occlusion device 200 includes a seal 224 for forming a seal between filter holder 126 and occlusion device 200, between filter holder 126 and a secondary occlusion device 202 and sterilizing cabinet 102. The seal 224 comprises an edge portion 222 having a thickness sufficient to form a sealed interface between at least one of the sterilizing cabinet 102 and the filter holder 126 to prevent extraneous materials from exiting the container. In one configuration, the seal 224 is a gasket that is permanently affixed or integral with the occlusion device. In another configuration, the seal 224 is a gasket that is separable from the nonporous cover. In another configuration, the seal is a gasket that is coextensive with the nonporous cover. This seal 224 keeps extraneous materials from exiting the sterilizing cabinet 102 through vents 124, which are covered by the occlusion device. In one exemplary embodiment, the seal 224 is a silicone seal. Exemplary embodiments and features of the occlusion device 200, 202 having seal 224 are further described below with respect to FIG. 17A-FIG. 23 which features and configurations are intended to be included in the above-described exemplary embodiments.

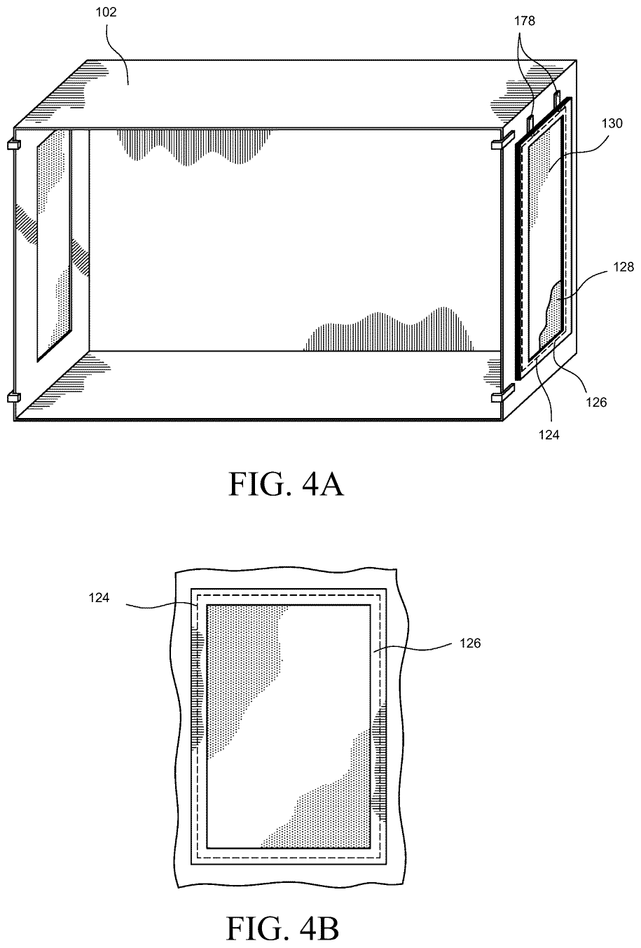

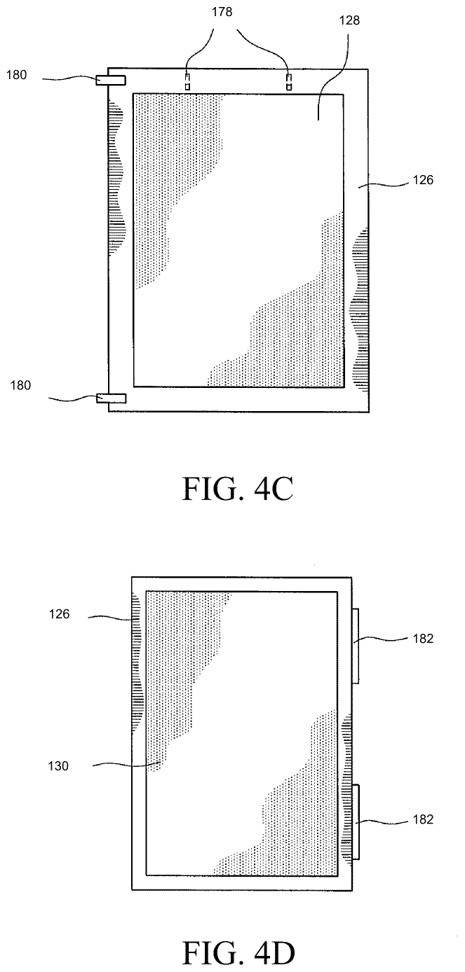

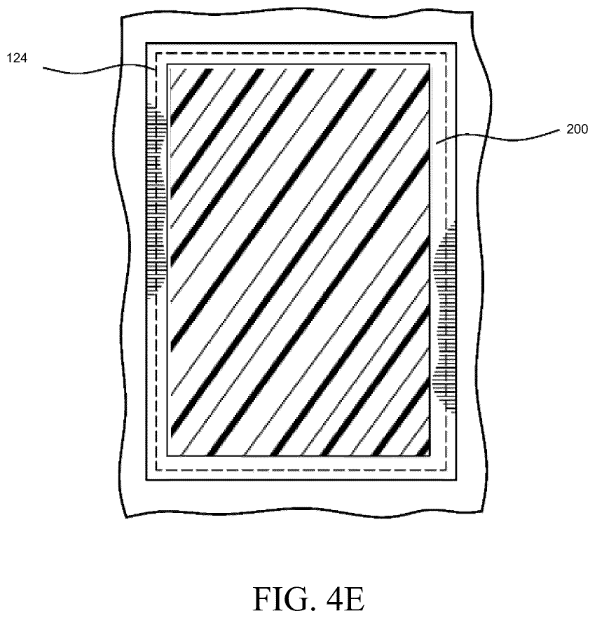

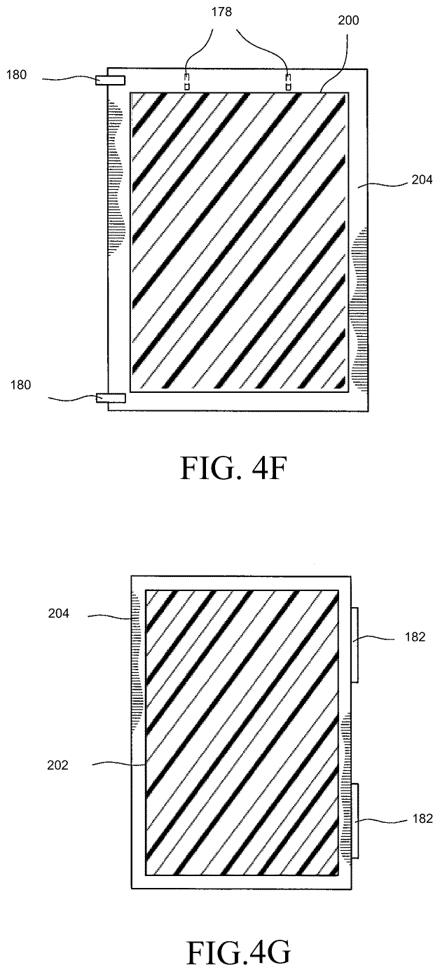

FIGS. 4A-4D provide a perspective view of alternative arrangements of sterilizing cabinet 102 in a sterilization configuration with vents 124 on the sides of the cabinet 102. In this embodiment filter holders 126 are located on the sides of sterilizing cabinet 102 with primary filter 128 and secondary filter 130. Also shown in FIG. 4A are hangers 178 from which filter holder 126, primary filter 128 and secondary filter 130 attach to sterilizing cabinet 102. It can be appreciated that exemplary embodiments of sterilizing cabinet 102 include vents 124, primary filter 128, secondary filter 130 and filter holder 126 on the side of sterilizing cabinet 102. In an exemplary embodiment, the filter holder 126 overlays vent 124 on sterilizing cabinet 102. In this embodiment filter holder 126 is sized such its edges completely cover the portions surrounding vent 124. FIG. 4B shows a front facing view of vent 124 with filter holder 126 overlaying vent 104 on the sterilizing cabinet 102. The edges of the filter holder 126 completely cover the portions on the sterilizing cabinet 102 covering vents 124. FIG. 4C shows primary filter 128 and filter holder 126 overlaying vent 104. Primary filter 128 as shown hangs from hangers 178 and attaches to filter holder 126 by securing mechanism 180, such as at least one clamp, latch or hinge. FIG. 4D shows secondary filter 130 in filter holder 126 overlaying primary filter 128 and vent port 124. In this configuration, the secondary filter 110 can be opened and closed through a securing mechanism 180, such as a hinge and pin mechanism.

In the transportation configuration of the sterilizing cabinet 102 of FIG. 4A, the filter holder 126 holds an occlusion device 200 and/or 202 instead of primary filter 128 and/or secondary filter 130 as shown in FIGS. 4E-4G . Alternatively, an occlusion device holder 204 is utilized in place of the filter holder 126. In one configuration, the occlusion device 200, either directly or via the filter holder 126 or occlusion device holder 204, is attached to the cabinet 102 by hangers 178. Additionally, the occlusion device 200, either directly or via the filter holder 126 or occlusion device holder 204, attaches to the sterilizing cabinet 102 by securing mechanism 180, such as at least one clamp, latch or hinge. The occlusion device 200 can additionally or alternatively be attached to the cabinet 102 by at least one clamp, latch or hinge. The occlusion device 200, 202 is a sheet comprising a nonporous material. In another configuration, the occlusion device 200, 202 is a cover having a thickness and rigidity for occluding the vent ports 124. By rigidity it is meant that the occlusion device is capable of holding its shape without further support. The occlusion device 200 in one configuration forms a sealed interface with an adjacent portion of the at least one of the cabinet 102 and the filter holder 126 or occlusion device holder 204 such that during the transportation of the cabinet 102 having at least one object 190, which can include at least one tray 160 (for example as shown in FIG. 12), the cabinet 102 is sealed to provide a leak-resistant transport system that contains contamination within the cabinet 102. In addition to being leak-resistant, the occlusion device 200 is tear and puncture resistant, thereby reducing the risk of injury and further containing the contaminated object(s) 190. In an exemplary embodiment, the occlusion device 200 is single-use and disposable after one use. In another exemplary embodiment, the occlusion device 200 is re-usable and washable along with the contaminated instruments. In certain exemplary embodiments, the occlusion device 200 is sterilizable. In a configuration, the occlusion device 200 includes a seal or gasket 224 (for example as shown in FIGS. 17A-23) for forming a seal between the sterilizing cabinet 102 and the filter holder 126 for occlusion device 200. The occlusion device 202 may include a seal 224 for forming a seal between the filter holder 126 for occlusion device 200 and the filter holder 126 for occlusion device 202. The occlusion device 202 may additionally or alternatively include a seal 224 for forming a seal with the sterilizing cabinet 102. The seal 224 in a configuration comprises an edge portion 222 having a thickness sufficient to form a sealed interface between at least one of the sterilizing cabinet 102 and the filter holder(s) 126 to prevent extraneous materials from exiting the container. In one configuration, the seal 224 is a gasket that is permanently affixed or integral with the occlusion device. In another configuration, the seal 224 is a gasket that is separable from the nonporous cover. In another configuration, the seal 224 is a gasket that is coextensive with the nonporous cover. This seal 224 keeps extraneous materials from exiting the sterilizing cabinet 102 through vents 124, which are covered by the occlusion device 200 and/or 202. In one exemplary embodiment, the seal 224 is a silicone seal. Exemplary embodiments and features of the occlusion device 200, 202 having seal 224 are further described below with respect to FIG. 17A-FIG. 23 which features and configurations are intended to be included in the above-described exemplary embodiments.

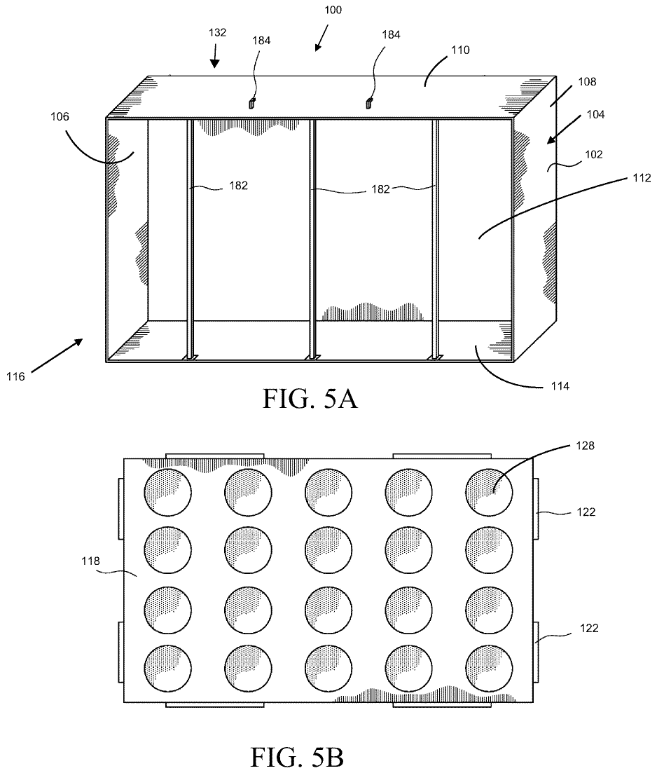

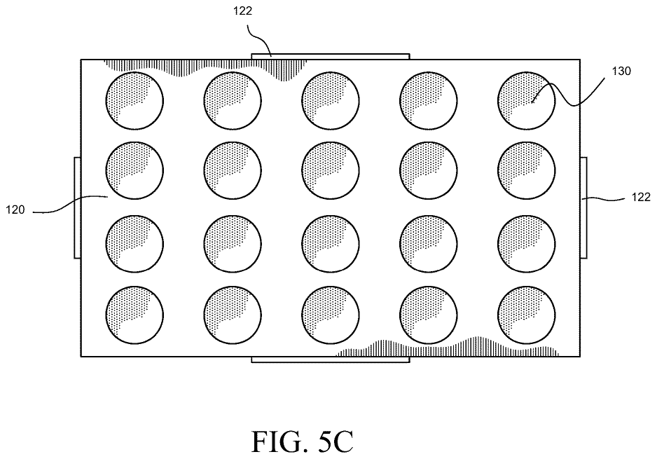

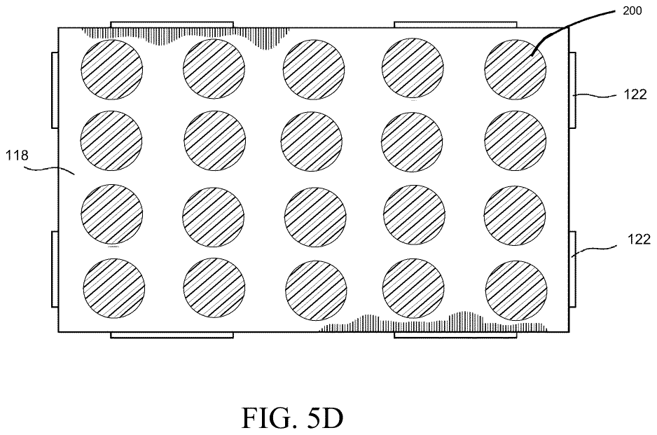

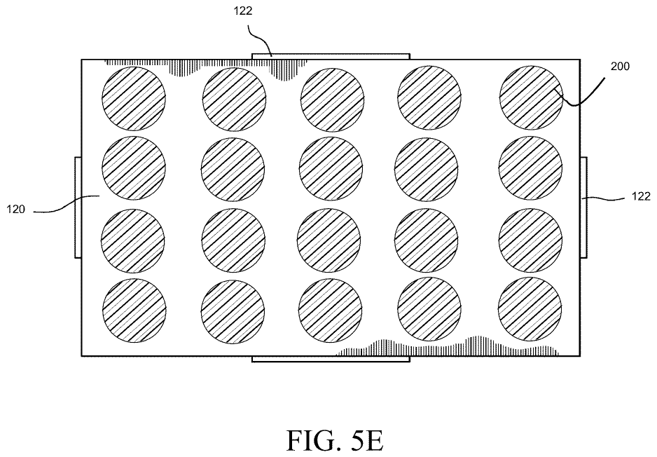

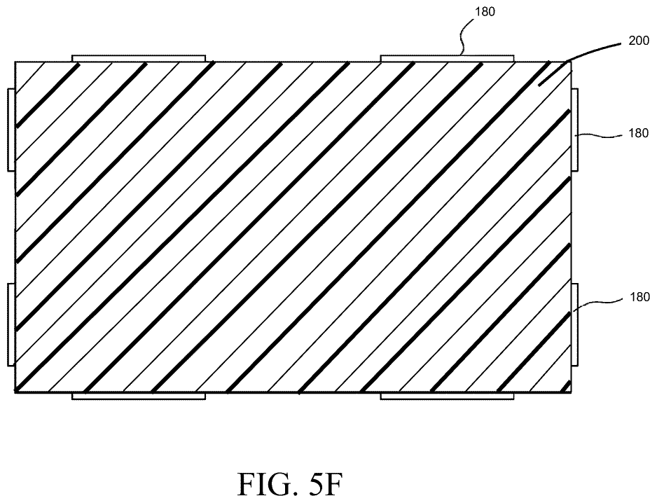

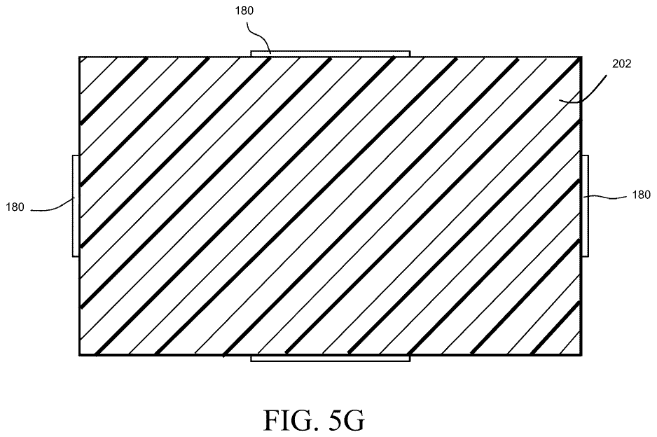

Turning now to FIGS. 5A-5F , FIG. 5A provides a perspective view of an alternative sterilizing container 100 for sterilizing and transporting objects 190. Shown in FIG. 5A is bars 182 and hooks 184. More specifically, the sterilizing cabinet 102 includes an enclosing wall 104 including sidewalls 106, 108, top wall 110, rear wall 112 and bottom wall 114 forming a sterilizing cabinet frame 132. An access port 116 can be lying in a plane and spaced from the rear wall 112. The access port 116 is sized to pass a plurality of trays, each tray sized to retain a plurality of objects. In this embodiment there is no front side of sterilizing container 100 in front of bars 182. It should be appreciated that exemplary embodiments of sterilizing container 100 also include embodiments of sterilizing container 100 that do not contain bars 182. In yet another exemplary embodiment of sterilizing container 100, bars 182 are removeable such that bars 182 can be removeably affixed to sterilizing container 100 when desired. FIG. 5B illustrates door 118 which is configured to contain primary filter 128. In exemplary embodiments filter door 118 covers the front opening of sterilizing container 100 in FIG. 5A. Filter door 118 clamps onto sterilizing container 100 with clamps 122. Bars 182 prevent the contents of sterilizing container 100 (typically a tray containing instruments for sterilization) from ripping or breaking primary filter 128 and, if applied, secondary filter 130 as shown in FIG. 5C.

FIG. 5C illustrates filter door 120 which attaches to filter door 118 and sterilizing container 100 with the use of clamps 122. In this embodiment it can be appreciated that clamps 122 on filter door 120 fit into the spacing between clamps 122 on filter door 118. This arrangement prevents the clamps 122 from filter door 118 from interfering with clamps 122 from filter door 118. Additionally, since filter door 120 is attached independently from filter door 118, secondary filter 130 can be removed with filter door 120 after a sterilization cycle has completed without disturbing filter door's 118 seal with sterilizing container 100. In this embodiment filter door 118 forms a seal with sterilizing container 100 at the edges of the open portion of the sterilizing container frame 132, such that any sterilizing steam that enters or exits sterilizing container 100 during a sterilization cycle must pass through filter door 118 and primary filter 128. Likewise, filter door 120 forms a seal with filter door 118 such that any sterilizing steam that exits sterilizing container 100 and primary filter 128 must pass through filter door 120 and secondary filter 130.

As shown in FIGS. 5D and 5E, in the exemplary embodiment a transportation configuration includes the sterilizing cabinet 102 of FIG. 5A, the door 118 and an occlusion device 200 that overlies the access port 116 instead of primary filter 128 and/or secondary filter 130. In one configuration, door 118 is configured to contain occlusion device 200. The occlusion device 200 in one configuration is attached to the cabinet 102 directly by securing mechanism 180, such as at least one clamp, latch or hinge. In one exemplary embodiment, the occlusion device 200 forms a sealed interface with an adjacent portion of at least one of the sterilization cabinet 102 and the door 118. In certain configurations, a second occlusion device 202 is included. For example, door 120 can be configured to contain occlusion device 202 or the occlusion device 202 can be attached to the outside of door 118. Alternatively, as shown in FIG. 5F, the occlusion device 200 overlies the access port 116 instead of the primary filter 128 and door 118. In this embodiment, the occlusion device 200 is attached to the cabinet 102 directly by securing mechanism 180, such as at least one clamp, latch or hinge. The occlusion device 200 forms a sealed interface with an adjacent portion of the sterilization cabinet 102. In certain configurations, a second occlusion device 202 is included as shown in FIG. 5G. For example, occlusion device 202 can be configured to overlie occlusion device 200 and form a seal with occlusion device 200. For example, occlusion device 202 can form a seal proximate a perimeter edge of the occlusion device 200.

As described above, the occlusion device 200, 202 is a sheet comprising a nonporous material. In another configuration, the occlusion device 200, 202 is a cover having a thickness and rigidity for occluding the access port 116. By rigidity it is meant that the occlusion device is capable of holding its shape without further support. The occlusion device 200 in one configuration forms a sealed interface with an adjacent portion of the at least one of the cabinet 102, and if included the filter door 118 or such that during the transportation of the cabinet 102 having at least one object 190, which can include at least one tray 160 (for example as shown in FIG. 12), the cabinet 102 is sealed to provide a leak-resistant transport system that contains contamination within the cabinet 102. In addition to being leak-resistant, the occlusion device 200 is tear and puncture resistant, thereby reducing the risk of injury and further containing the contaminated object(s) 190. In an exemplary embodiment, the occlusion device 200 is single-use and disposable after one use. In another exemplary embodiment, the occlusion device 200 is re-usable and washable along with the contaminated instruments. In certain exemplary embodiments, the occlusion device 200 is sterilizable. In a configuration, the occlusion device 200 includes a seal or gasket 224 (for example as shown in FIGS. 17A-23) for forming a seal between the sterilizing cabinet 102 and the filter door 118 for occlusion device 200. The occlusion device 202 may include a seal 224 for forming a seal between the filter door 118 for occlusion device 200 and the filter door 120 for occlusion device 202. The occlusion device 202 may additionally or alternatively include a seal 224 for forming a seal with the sterilizing cabinet 102. The seal 224 in a configuration comprises an edge portion 222 having a thickness sufficient to form a sealed interface between at least one of the sterilizing cabinet 102 and the filter door(s) 118, 120 to prevent extraneous materials from exiting the container. In one configuration, the seal 224 is a gasket that is permanently affixed or integral with the occlusion device. In another configuration, the seal 224 is a gasket that is separable from the nonporous cover. In another configuration, the seal 224 is a gasket that is coextensive with the nonporous cover. This seal 224 keeps extraneous materials from exiting the sterilizing cabinet 102 through vents 124, which are covered by the occlusion device 200 and/or 202. In one exemplary embodiment, the seal 224 is a silicone seal. Exemplary embodiments and features of the occlusion device 200, 202 having seal 224 are further described below with respect to FIG. 17A-FIG. 23 which features and configurations are intended to be included in the above-described exemplary embodiments.

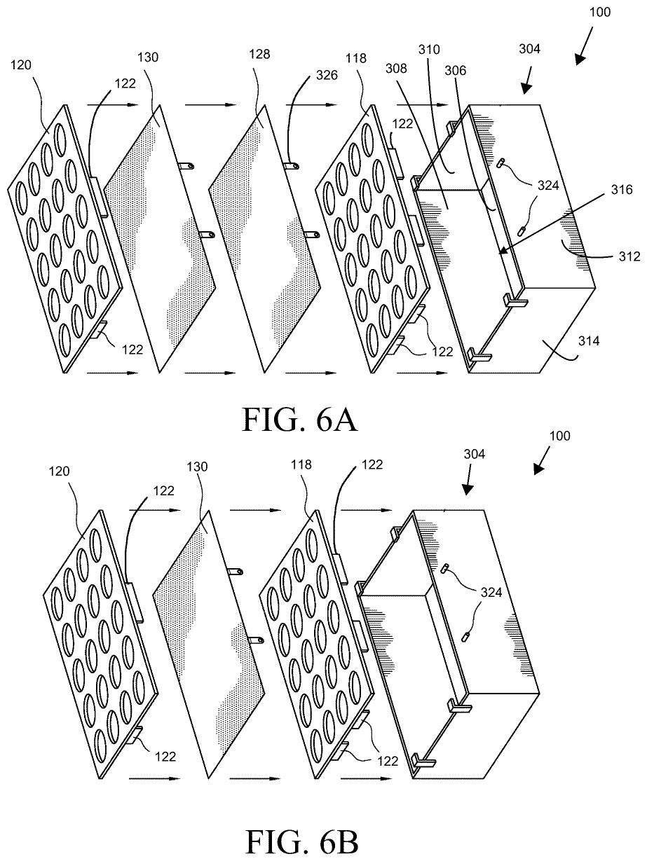

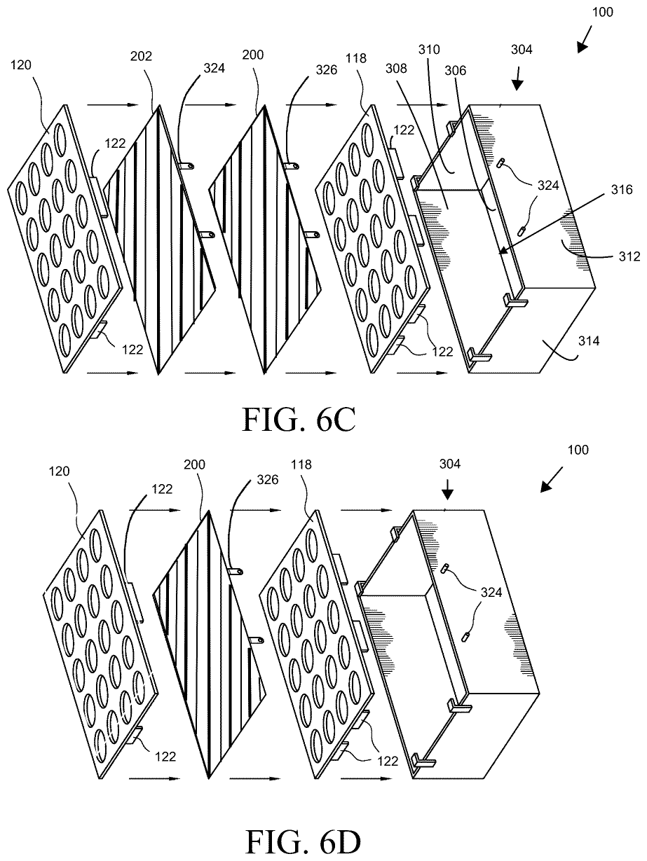

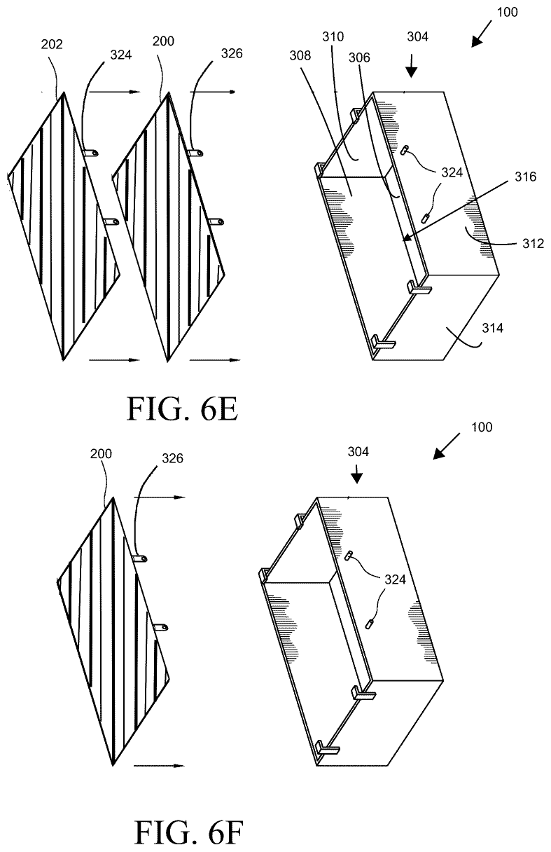

FIGS. 6A and 6B depict perspective views of the construction of an alternative arrangement sterilizing container 100 from FIGS. 5A, 5B and 5C. In this configuration, the container 100 comprises an enclosing wall 304 having a bottom wall 306 and sidewalls 308, 310, 312, and 314, wherein the access port 316 is lying in a plane spaced from the bottom wall 306 of the enclosing wall 304.

As shown in FIG. 6A, filter door 118 with clamps 122 attach around the frame of sterilizing container 100. Primary filter 128 is placed on top of filter door 118 and attaches to sterilizing container 100 at hooks 324. Secondary filter 130 is placed on top of primary filter 128 and also attaches to sterilizing container 100 at hooks 324. Filter door 120 is then placed on top of secondary filter 130 and attached to sterilizing container 100 with clamps 122. As illustrated in FIG. 6A, exemplary embodiments of filter doors 118 and 120 contain numerous holes or openings along their surface, and allow for the passage of sterilizing steam. Exemplary embodiments of filter doors 118 and 120 are able to be fully or partially separable from sterilizing container 100. It should also be appreciated that filter doors 118 and 120 can optionally employ the use of a hinge, clamp, clasp or the like as the mechanism for removing and replacing filter doors 118 and 120 on sterilizing container 100.

FIG. 6B depicts an alternative perspective view of the construction of another alternative arrangement sterilizing container 100. As shown filter door 118 with clamps 122 attach around the frame of sterilizing container 100. Primary filter 128 is placed on top of filter door 118 and attaches to sterilizing container 100 at hooks 324. In this embodiment, there is only a single filter and no secondary filter. Primary filter 128 creates a sealed interface with filter door 118 such that extraneous materials cannot enter sterilizing container 100. Filter door 120 is then placed on top of primary filter 128 and attached to sterilizing container 100 with clamps 122. In another exemplary embodiment, primary filter 128 does not create a sealed interface with filter door 118 until filter door 120 is placed on top of primary filter 128 and filter door 118. In this embodiment, a sealed interface between filter door 120 and primary filter 128, and a sealed interface between filter door 118 or sterilizing container 100 and primary filter 128 is only created when filter door 120 is attached or affixed to sterilizing container 100. As illustrated in FIG. 6B, exemplary embodiments of filter doors 118 and 120 which contain numerous holes or openings along their surface, which allow for the passage of sterilizing steam.

It should be noted that exemplary embodiments of FIG. 6B provide that filter door 120 can be removed from sterilizing container 100, primary filter 128 and filter door 118 without disturbing or disrupting the sealed interface between primary filter 128 and filter door 118. This will prevent the possibility of extraneous materials from entering sterilizing container 100 after a sterilizing cycle when filter door 118 is removed in order to either allow primary filter 128 and filter door 118 to be removed as well or for primary filter 128 to be inspected to verify that it maintained its integrity during the sterilizing cycle.

In an alternative exemplary embodiment, the sealed interface between filter door 120 and primary filter 128, and the sealed interface between filter door 118 or sterilizing container 100 and primary filter 128 is broken or can be broken when filter door 120 is removed from sterilizing container 100, primary filter 128, and filter door 118. In yet another exemplary embodiment, the sealed interface between filter door 120 and primary filter 128, and the sealed interface between filter door 118 or sterilizing container 100 and primary filter 128 is only created and thereafter maintained when sterilizing container 100 along with filter door 120, filter door 118 and primary filter 128 are exposed to a sterilization cycle. Exemplary embodiments of sterilizing container 100 as depicted in FIG. 6B are able to maintain the sealed interface between filter door 120 and primary filter 128, and the sealed interface between filter door 118 or sterilizing container 100 and primary filter 128 for an extended period of time following being exposed to a sterilization cycle, such as sterilizing steam. For example, the sealed interface can be able to remain intact for as long as 30-90 days. In other exemplary embodiments the sealed interface can only remain intact for a matter of hours.

In the exemplary embodiment shown in FIGS. 6C and 6D, a transportation configuration includes the sterilizing container 100, door 118, door 120 as shown in FIGS. 6A and 6B and at least one occlusion device 200 that overlies the access port 316 instead of primary filter 128 and/or secondary filter 130. In one configuration, the occlusion device 200 is attached to the enclosing wall 304 directly by attaching tabs 326 to securing mechanism, such as at least one clamp, latch, hinge, or hooks 324. The filter door 118 with clamps 122 attaches around the frame of sterilizing container 100. The occlusion device 200 is placed on top of filter door 118 and attaches to sterilizing container 100 at hooks 324 wherein the occlusion device 200 creates a sealed interface with filter door 118 such that extraneous materials cannot enter sterilizing container 100. In some embodiments, the filter door 120 can be placed on top of the occlusion device 200 and attached to sterilizing container 100 with clamps 122. This can avoid losing the filter door 120 during transportation since it keeps all the elements of the sterilizing container 100 together.

However, it should be appreciated that in certain configurations, the occlusion device 200 does not require the addition of the filter door 120 to form a seal with the container.

In certain exemplary embodiment, the occlusion device 200 does not create a sealed interface with filter door 118 until filter door 120 is placed on top of the occlusion device 200 and filter door 118. In this embodiment, a sealed interface between filter door 120 and the occlusion device 200 and a sealed interface between filter door 118 or sterilizing container 100 and occlusion device 200 is only created when filter door 120 is attached or affixed to sterilizing container 100.

As shown in FIGS. 6E and 6F, occlusion device 200 attaches directly to enclosing wall 304 wherein a sealed interface is formed with the perimeter edge of the container 100. For example, in one configuration, tabs 326 attached to securing mechanism 324.

In certain configurations, as shown in FIGS. 6C and 6E, a second occlusion device 202 is included in the transportation configuration. For example, in place of filters 128 and 130 shown in FIG. 6A, occlusion device 200 and occlusion device 202 can be attached to the outside of door 118 as shown in FIG. 6C. Alternatively, as shown in FIGS. 6E and 6F, the occlusion device 200 overlies the access port 116 instead of the primary filter 128, secondary filter 130, and doors 118, 120. As shown in FIG. 6F, in this embodiment, the occlusion device 200 includes tabs 326 that attach to the container 100 directly by a securing mechanism, such as at least one clamp, latch, hinge or hook 324. As shown in FIG. 6E, occlusion device 202 also includes tabs 326 that attached to the container 100 directly by a securing mechanisms, such as at least one clamp, latch, hinge or hook 324. In this configuration, the doors 118, 120 can be transported separately from the sterilizing container 100. The occlusion device 200 forms a sealed interface with an adjacent portion of the sterilization cabinet 102. In certain configurations, a second occlusion device 202 is included as shown in FIG. 5G. For example, occlusion device 202 can be configured to overlie occlusion device 200 and form a seal with occlusion device 200. For example, occlusion device 202 can form a seal proximate a perimeter edge of the occlusion device 200.

As described above, the occlusion device 200, 202 is a sheet comprising a nonporous material. In another configuration, the occlusion device 200, 202 is a cover having a thickness and rigidity for occluding the access port 116. By rigidity it is meant that the occlusion device is capable of holding its shape without further support. The occlusion device 200 in one configuration forms a sealed interface with an adjacent portion of the at least one of the container 100, and if included the filter door 118 or such that during the transportation of the container 100 having at least one object 190, which can include at least one tray 160 (for example as shown in FIG. 12), the container 100 is sealed to provide a leak-resistant transport system that contains contamination within the container 100. In addition to being leak-resistant, the occlusion device 200 is tear and puncture resistant, thereby reducing the risk of injury and further containing the contaminated object(s) 190. In an exemplary embodiment, the occlusion device 200 is single-use and disposable after one use. In another exemplary embodiment, the occlusion device 200 is re-usable and washable along with the contaminated instruments. In certain exemplary embodiments, the occlusion device 200 is sterilizable. In a configuration, the occlusion device 200 includes a seal or gasket 224 (for example as shown in FIGS. 17A-23) for forming a seal between one of the container 100 and the filter door 118. The occlusion device 202 may include a seal 224 for forming a seal between the filter door 118 and the filter door 120 in the configuration shown in FIG. 6C. The occlusion device 202 may include a seal 224 for forming a seal between occlusion device 200 and occlusion device 202 in the configuration shown in FIG. 6E. The occlusion device 202 may additionally or alternatively include a seal 224 for forming a seal with the sterilizing container 100. The seal 224 in a configuration comprises an edge portion 222 having a thickness sufficient to form a sealed interface between at least one of the sterilizing container 100 and the filter door(s) 118, 120 to prevent extraneous materials from exiting the container. In one configuration, the seal 224 is a gasket that is permanently affixed or integral with the occlusion device. In another configuration, the seal 224 is a gasket that is separable from the nonporous cover. In another configuration, the seal 224 is a gasket that is coextensive with the nonporous cover. This seal 224 keeps extraneous materials from exiting the sterilizing container 100 through the access port 316, which is covered by the occlusion device 200 and/or 202. In one exemplary embodiment, the seal 224 is a silicone seal. Exemplary embodiments and features of the occlusion device 200, 202 having seal 224 are further described below with respect to FIG. 17A-FIG. 23 which features and configurations are intended to be included in the above-described exemplary embodiments.

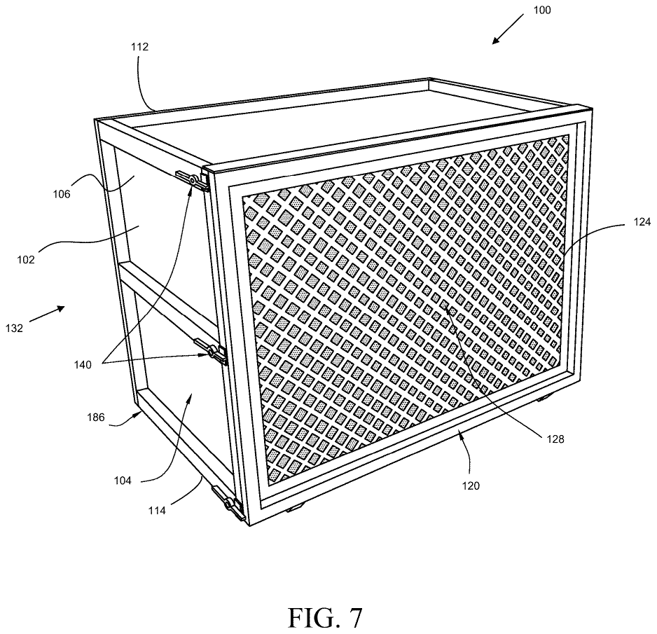

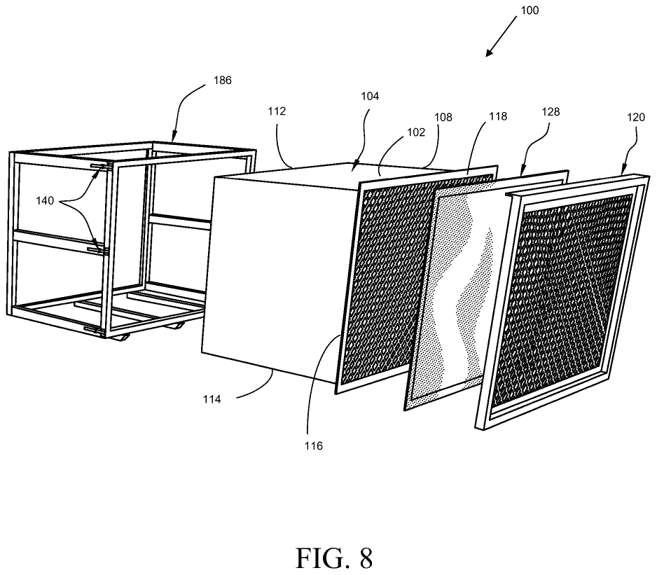

FIGS. 7 and 8 show a perspective view of alternative embodiments of a sterilizing cabinet 100 suitable for use in practicing exemplary embodiments of this disclosure. As shown in FIG. 1, the sterilizing container can be a sterilizing cabinet 102 having an enclosing wall 104 including sidewalls 106, 108, top wall 110, rear wall 112 and bottom wall 114 forming a sterilizing cabinet frame 132. An access port 116 can be lying in a plane and spaced from the rear wall 112. The access port 116 is sized to pass a plurality of trays 160, each tray 160 sized to retain a plurality of objects 190. Sterilizing container 100 further includes vent 124, and latches 140. In certain configurations, the sterilizing cabinet 102 is maintained within frame 186. Frame 186 provides a structurally reinforcing frame for sterilizing cabinet 102. Door 118 or doors 118, 120 can be removable from sterilizing cabinet 102. It should be appreciated that exemplary embodiments of door 118 or doors 118, 120 include any mechanism that allows for door 118 or doors 118, 120 to move from an open position to a closed position to provide access to the interior of sterilizing cabinet 102. In this configuration, the vent 124 is on the door. The vent 124 provide numerous small openings for the passage of sterilizing steam. The small openings in vents 124 can be holes or slits. Alternatively, vent 124 can be fenestrated. A fenestrated vent surface with numerous openings allows for the passage of a sterilizing agent, such as steam from an autoclave during a sterilization cycle.

Referring to FIG. 8, shown is a perspective view of a separated alternative embodiment of a sterilizing container suitable for use in practicing exemplary embodiments of this disclosure. Shown in FIG. 8 are sterilizing container 100, frame 186, filter door 118, filter door 120, primary filter 128, which can be a filter cartridge, and latches 140. In some embodiments, as shown in FIG. 8, sterilizing container 100 can be removed from frame 186. In other exemplary embodiments, frame 186 is not removable from sterilizing container 100, but is fixedly attached to sterilizing container 100. Filter door 120 as depicted includes a fenestrated grid throughout its center. However, exemplary embodiments of filter door 120 include any type of arrangement of holes, gaps, or grids such that sterilizing steam is free to pass through the center portion of filter door 118, 120.

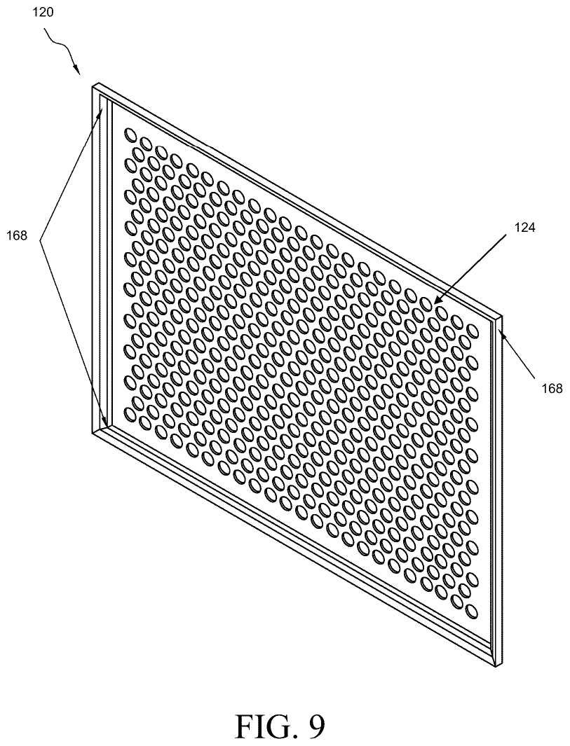

Referring to FIG. 9, shown is an exemplary filter door 120 for sterilizing container 100. In one configuration, filter door 120 is an outer filter door. Filter door 120 includes tabs or catches 168, which protrude perpendicular from the back face of filter door 120. Filter door 120 as illustrated in FIG. 9 contains a vent port 124 which comprises numerous holes or openings along its surface, which allow for the passage of sterilizing steam. Tabs or catches 168 for removably locating or placing a filter or filter cartridge on filter door 120. Tabs or catches 168 can be located in each of the four corners of filter door 120. However, it should be noted that in other exemplary embodiments there can be more or less than four tabs or catches 168 and in different arrangements.

In one exemplary embodiment, during a sterilizing cycle primary filter 128 is maintained between filter door 120 and sterilizing container 100 creating a sealed interface around its edges such that sterilizing steam is not able to pass between the sealed interface, but can only pass through the center of filter 128. In certain configurations, a second filter is placed between filter door 120 and the sterilizing container 100. In yet another configuration, the sterilizing container 100 includes a second door such that a primary filter 128 (not shown) is maintained between filter door 118 and sterilizing container 100 and a secondary filter 130 is maintained between the filter door 118 and the second filter door 120.

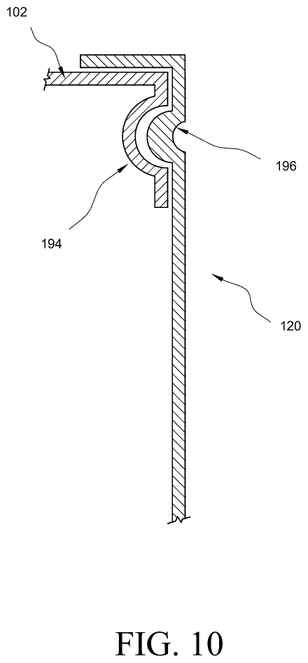

Referring to FIG. 10, shown is a magnified cross-sectional view of an exemplary sterilizing container and filter door shown with trough section 194, and filter door 120 with trough section 196. Exemplary embodiments of trough section 194 and trough section 196 run along the entire edge of sterilizing container 100 and filter door 120. Trough section 194 and trough section 196 are shaped such that a sealed interface is maintained throughout the trough (i.e., between trough section 194 and trough section 196) when a single filter, multiple filters, or a filter cartridge is placed between trough section 194 and trough section 196. Exemplary embodiments of trough section 194 and trough section 196 have a size and depth such that the movement of extraneous materials through the sealed interface between sterilizing container 100 and filter door 120 is substantially prevented. In practice, a filter cartridge can be placed and compressed between the sterilizing container 100 and filter door 120 such that the filter cartridge is deformed into the shape of the trough in which it is compressed. In the transportation configuration, an occlusion device 200 is placed and compressed between the sterilizing container 100 and the filter door 120 such that the occlusion device is deformed into the shape of the trough in which it is compressed and forms a seal to prevent leakage of the contamination contained within the container 100.

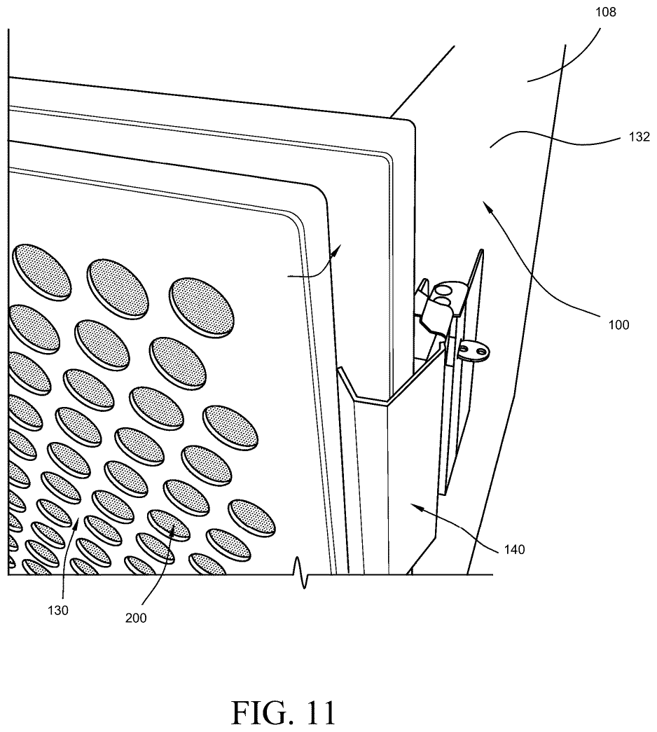

FIG. 11 shows clamp 140 located on the vertical sides of sterilizing container 100. However, exemplary embodiments of clamp 140 can be placed in many different arrangements along the sides of sterilizing container 100 such that clamps 140 are able to clasp and maintain filter door 130 in a sealed position over the open front of sterilizing container 100. Exemplary embodiments of clamps 140 are able to clasp and release filter door 130 from sterilizing container 100. Exemplary embodiments of clamp 140 are sized such that a sealed interface is created between sterilizing container 100, a filter or filters, and the filter doors. In certain configurations, the clamp 140 is adjustable to accommodate different thicknesses between the filter(s) 128, 130 and the occlusion device(s) 200 and 202. The sealed interface prevents the passage of extraneous materials between the filter doors and sterilizing container 100. Moreover, exemplary embodiments of clamp 140 are sized such that a sealed interface is created between sterilizing container 100, an occlusion device 200 or devices 202, and the filter door 118 or 120. The sealed interface prevents liquid, gases, and other contaminated materials from the objects within the container 100 from leaking between the filter door 118 or filter doors 118, 120 and sterilizing container 100. While filter door 130 attaches to frame 132 through the use of latches 140, it should be appreciated that latches 140 can include any type of clamping, latching or clasping device known in the art that is able to releasable attach filter door 130 to frame 132 such that a sealed interface is created between filter door 130 and sterilizing container 100.

FIG. 12 shows an exploded view of the sterilizing container 100 having a configuration similar to that shown in FIGS. 5A-5E, 7-11 in a transportation configuration. The container 100 is a sterilizing cabinet 102 having an enclosing wall 104 including sidewalls 106, 108, top wall 110, rear wall 112 and bottom wall 114 forming a sterilizing cabinet frame 132. An access port 116 can be lying in a plane and spaced from the rear wall 112. The sterilizing container 100 further includes a door 118 connected to the container 100. In one configuration the door 118 is removable from the container 100. In another configuration, the door 118 is connected to the container 100 and not removeable. In either configuration, however, the door 118 is moveable between an open position permitting passage through the access port 116 to an interior of the container 100 and a closed position precluding passage through the access port 116. The door 118 includes a vent port 124. The vent port(s) 124 can comprise a plurality of holes or openings 188 that allow steam to penetrate into the container 100 in the sterilization configuration. In other configuration, the vent port(s) 124 comprises a fenestrated surface that allows steam to penetrate into the container in the sterilization configuration. In the transportation configuration, the occlusion device 200 overlies the vent port 124 to occlude the vent port(s) 124. No filter is included. In one configuration, the occlusion device 200 is a cover comprising a sheet 210 of a nonporous material. The sheet 210 may include a frame 208 for supporting the sheet 210. In another configuration, the occlusion device 200 is a cover having a thickness and rigidity for occluding the access port 116. By rigidity it is meant that the occlusion device is capable of holding its shape without further support. The occlusion device 200 in one configuration forms a sealed interface with an adjacent portion of the cabinet 102 such that during the transportation of the cabinet 102 having at least one object 190, which can include at least one tray 160, the cabinet 102 is sealed to provide a leak-resistant transport system that contains contamination within the cabinet 102. In addition to being leak-resistant, the occlusion device 200 is tear and puncture resistant, thereby reducing the risk of injury and further containing the contaminated object(s) 190. In an exemplary embodiment, the occlusion device 200 is single-use and disposable after one use. In another exemplary embodiment, the occlusion device 200 is re-usable and washable along with the contaminated instruments. In certain exemplary embodiments, the occlusion device 200 is sterilizable. In a configuration, the occlusion device 200 includes a seal or gasket 224 (for example as shown in FIGS. 17A-23) for forming a seal between the sterilizing cabinet 102 and the occlusion device 200. The seal 224 in a configuration comprises an edge portion 222 having a thickness sufficient to form a sealed interface between the sterilizing cabinet 102 and the occlusion device 200 to prevent extraneous materials from exiting the container. In one configuration, the seal 224 is a gasket that is permanently affixed or integral with the occlusion device. In another configuration, the seal 224 is a gasket that is separable from the nonporous cover. In another configuration, the seal 224 is a gasket that is coextensive with the nonporous cover. This seal 224 keeps extraneous materials from exiting the sterilizing cabinet 102 through the access port 116 and, if the door is affixed to the cabinet 102 in the transportation configuration, also vents 124, which are covered by the occlusion device 200. In one exemplary embodiment, the seal 224 is a silicone seal. Exemplary embodiments and features of the occlusion device 200 having seal 224 are further described below with respect to FIG. 17A-FIG. 23 which features and configurations are intended to be included in the above-described exemplary embodiments.

As discussed above, in some embodiments, the filter door 118 can be placed over the occlusion device 200 and attached to sterilizing container 100 with clamps (not shown). This can avoid losing the filter door 118 during transportation since it keeps all the elements of the sterilizing container 100 together. However, it should be appreciated that the occlusion device 200 in certain embodiments does not require the addition of the filter door 118 to form a seal with the container 100. Instead, the occlusion device 200 is secured directly to the container 100 using clamps or latches, for example latches 140 as shown in FIGS. 7, 8, and 11 In another exemplary embodiment, the occlusion device 200 does not create a sealed interface with filter door 118 until filter door 118 is attached or affixed to the cabinet 102.

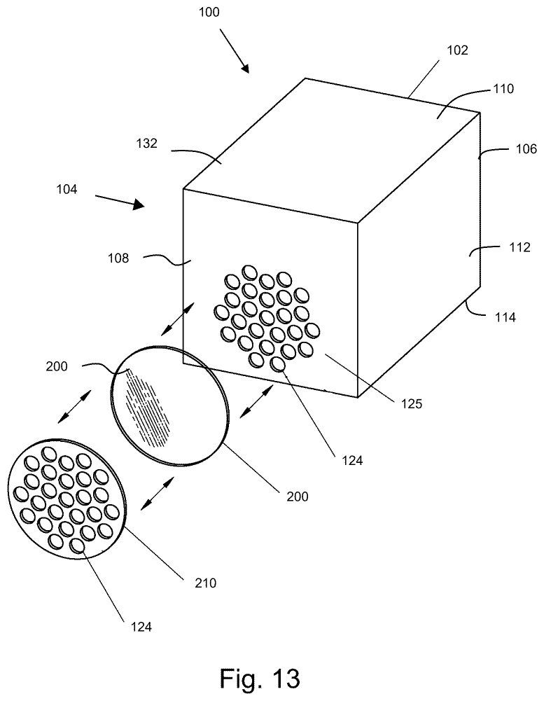

FIG. 13 shows an exploded view of the sterilizing container 100 in a transportation configuration and having features similar to FIGS. 1-4G in that the vent port(s) 124 are on the side of the sterilizing container 100. The sterilizing container 100 is a cabinet 102 have an enclosing wall 104 including sidewalls 106, 108, top wall 110, rear wall 112 and bottom wall 114 forming a sterilizing cabinet frame 132. An access port 116 (not shown) can be lying in a plane and spaced from the rear wall 112. The sterilizing container 100 further includes a door (not shown) connected to the container 100. In one configuration the door is removable from the container 100. In another configuration, the door is connected to the container 100 and not removeable. In either configuration, however, the door or doors are moveable between an open position permitting the placement of a plurality of trays or objects in the sterilizing container 100 through the access port. The sterilizing container 100 further includes a vent area 125 having vent ports 124 on the sidewalls 106, 108 or other walls, such as the top wall 110, rear wall 112 and bottom wall 114. The sterilizing container 100 can also include a vent door 210. In the transportation configuration, an occlusion device 200 is placed between vent area 125 and door 210. In this configuration, the door 210 can include vent ports 124 or be fenestrated to allow steam to penetrate into the container 100 in the sterilization configuration. In the transportation configuration, the occlusion device 200 overlies the vent area 125 to occlude the vent port(s) 124 and no filter is included therebetween. In the sterilization configuration, the occlusion device 200 is removed and replaced with at least one filter or a filter cartridge. In one configuration, the occlusion device 200 is a cover comprising a sheet of a nonporous material. The nonporous cover 200 in one configuration forms a sealed interface with an adjacent portion of the at least one of the container 100 and the door 210. Thus, when the door precluding access to the access port is closed and the occlusion device 200 is sealed to the cabinet 102, a leak-resistant transport system is provided that contains any contamination within the cabinet 102. In addition to being leak-resistant, the occlusion device 200 is tear and puncture resistant, thereby reducing the risk of injury and further containing the contaminated object(s) 190.

In another configuration, the occlusion device 200 is a cover having a thickness and rigidity for occluding the vent area 125 and vent ports on door 210. By rigidity it is meant that the occlusion device is capable of holding its shape without further support. In an exemplary embodiment, the occlusion device 200 is single-use and disposable after one use. In another exemplary embodiment, the occlusion device 200 is re-usable and washable along with the contaminated instruments. In certain exemplary embodiments, the occlusion device 200 is sterilizable. In a configuration, the occlusion device 200 includes a seal or gasket 224 (for example as shown in FIGS. 17A-23) for forming a seal between the sterilizing cabinet 102 and the door 210. The seal 224 in a configuration comprises an edge portion 222 having a thickness sufficient to form a sealed interface between at least one of the sterilizing cabinet 102 and the door 210 to prevent extraneous materials from exiting the container. In one configuration, the seal 224 is a gasket that is permanently affixed or integral with the occlusion device 200. In another configuration, the seal 224 is a gasket that is separable from the nonporous cover. In another configuration, the seal 224 is a gasket that is coextensive with the nonporous cover. This seal 224 keeps extraneous materials from exiting the sterilizing cabinet 102 through vents 124, which are covered by the occlusion device 200. In one exemplary embodiment, the seal 224 is a silicone seal. Exemplary embodiments and features of the occlusion device 200, 202 having seal 224 are further described below with respect to FIG. 17A-FIG. 23 which features and configurations are intended to be included in the above-described exemplary embodiments.

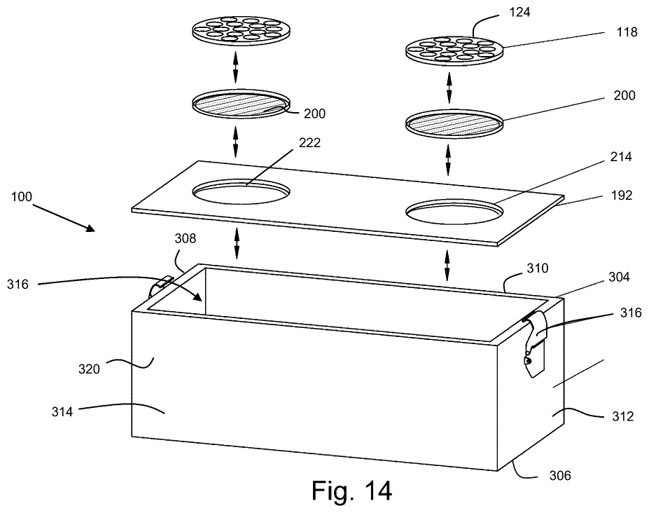

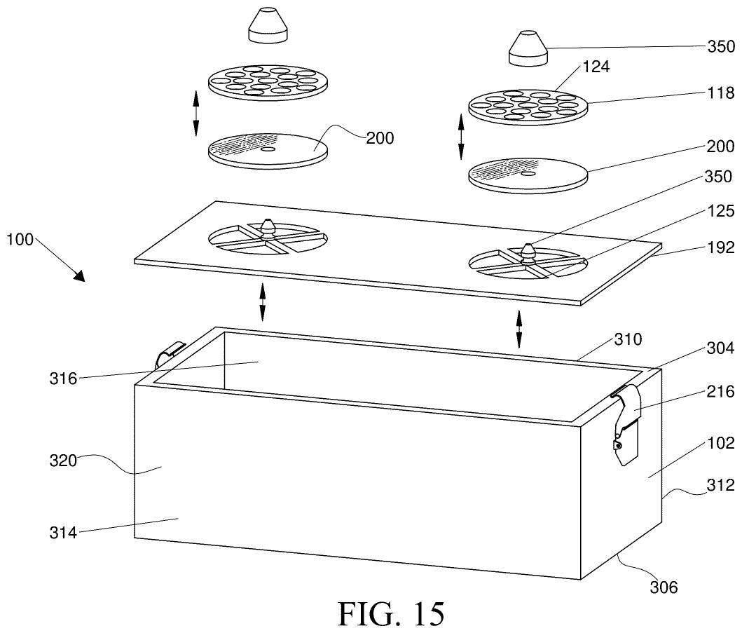

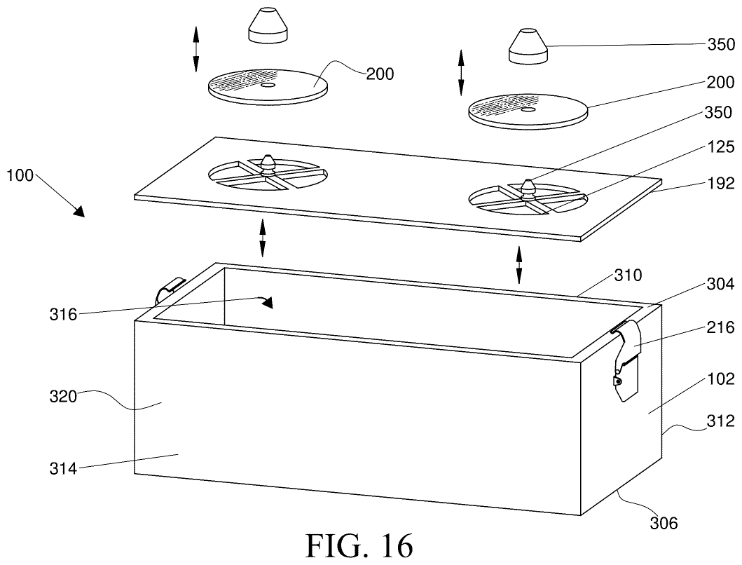

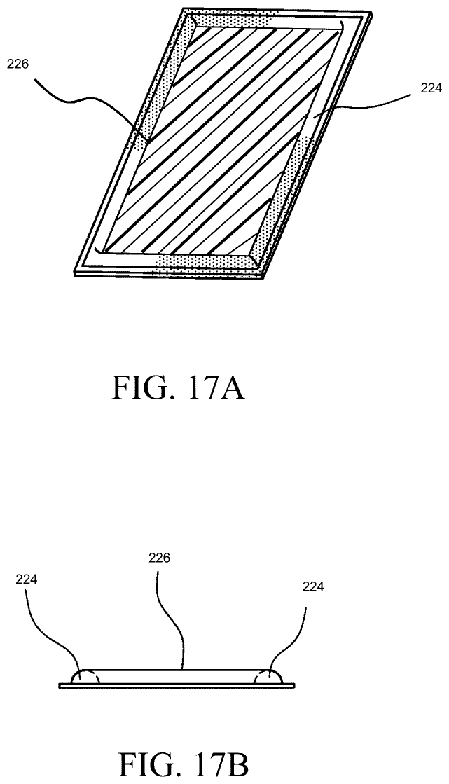

FIGS. 14, 15 and 16 show additional exemplary embodiments of the sterilizing container 100 in a transportation configuration. In this configuration, the container 100 includes a base 320 comprising an enclosing wall 304 having a bottom wall 306 and sidewalls 308, 310, 312, and 314, wherein the access port 316 is lying in a plane spaced from the bottom wall 306 of the enclosing wall 304. The sterilizing container 100 further includes a lid 192 connected to the container 100. In one configuration the lid 192 is removable from the container 100. In another configuration, the lid 192 is connected to the container 100 and not removeable. In either configuration, however, the lid 192 is moveable between an open position permitting passage through the access port 316 to an interior of the container 100 and a closed position precluding passage through the access port 316. The sterilizing container 100 can further include a screen or door 118 which includes a vent port or a plurality of vent ports 124. In another configuration, the container 100 includes a vent port area 125 as shown in FIGS. 15 and 16. The vent port 124 or vent port area 125 can comprise a plurality of holes or openings that allow steam to penetrate into the container 100 in the sterilization configuration. In other configuration, the vent port 124 or vent port area 125 comprises a fenestrated surface that allows steam to penetrate into the container in the sterilization configuration. Further, the lid 192 can include an opening 214 which can optionally include a plurality of holes that allow steam to penetrate into the container in the sterilization configuration. In another configuration, the opening is fenestrated as described above. In the transportation configuration, an occlusion device 200 overlies the vent port area 125 or opening 214 to occlude the vent port area 125 or opening 214 and no filter is included. As shown in FIG. 15, the door 118 can overlie the occlusion device 200 and then be attached or affixed to the lid 192 by locking mechanism 350 wherein the occlusion device 200 forms a seal between the lid 192 and the occlusion device 200 to prevent contaminants from exiting the container 100. Including the door 118 with the occlusion device 200 may avoid losing the filter door 118 during transportation since it keeps all the elements of the sterilizing container 100 together. However, as shown in the alternative configuration of FIG. 16, it should be appreciated that the occlusion device 200 in certain embodiments may not require the addition of the filter door 118 to form a seal with the container. The occlusion device 200 can, alternatively, be secured into place to form a seal between the lid 192 and the occlusion device 200 without the door 118 such that the contamination cannot exit the sterilizing container 100.

In one configuration, the occlusion device 200 is a cover comprising a sheet of a nonporous material. The nonporous cover 200 in one configuration forms a sealed interface with an adjacent portion of the lid 192. Clamps 216, when engaged, seal the lid 192 to the base 320 to provide a leak-resistant seal. During the transportation of the container having at least one contaminated object 190, the container 100 is sealed to provide a leak-resistant transport system that contains contamination within the container 100. In addition to being leak-resistant, the occlusion device 200 is tear and puncture resistant, thereby reducing the risk of injury and further containing the contaminated object(s) 190. In an exemplary embodiment, the occlusion device 200 is single-use and disposable after one use. In another exemplary embodiment, the occlusion device 200 is re-usable and washable along with the contaminated instruments. In certain exemplary embodiments, the occlusion device 200 is sterilizable.

For all configurations described, occlusion device 200 is made of nonporous material that is latex-free. By nonporous material it is meant to refer to any type of material that has no pores and prevents contaminants from exiting the container. The nonporous material is solid and non-fenestrated. Moreover, the nonporous material does not allow water droplets to pass. In a configuration, the nonporous material resists punctures or tears. In some configurations, the nonporous material is leak resistant. In other configurations, the nonporous material is leakproof. The nonporous material forms a barrier that does not allow liquids or gasses to pass through the material. In certain configurations, the nonporous material does not allow steam or gas to pass through. This material can include, but not be limited to any of, or a combination of plastic, glass, acrylic solids, metals, and vinyl. In one configuration, the nonporous material is silicone rubber. In another configuration, the nonporous material is polyurethane. The nonporous material can have a thickness ranging from approximately 0.0005 (12.7 microns) inches to approximately 3 inches. In some configurations, the nonporous material has a thickness of approximately 8-10 microns. In other configurations, the material has a thickness of approximately 0.35 mm-0.5 mm. In certain configurations the nonporous material is sized to have a thickness that corresponds to the thickness of the filter, filters, or filter cartridges used in the sterilization configuration. In certain configurations, the nonporous material is sized to have a thickness that is substantially the same as the thickness of the filter, filters or filter cartridges used in the sterilization configuration. In certain configurations, securing mechanisms that secure the occlusion device 200 to the sterilizing container 100 are adjustable to accommodate the varying thickness between the type of filters used in the sterilization configuration compared to the thickness of the occlusion device 200 used in the transportation configuration. The nonporous cover 200 can include one or more layers of material. In one configuration, the nonporous cover is two-ply. In another configuration, the nonporous cover is three-ply. In certain configurations, the occlusion device 200 is rigid. By rigid it is meant that the occlusion device 200 is capable of maintaining its shape. Alternatively, the occlusion device is semi-rigid. In yet another configuration, the occlusion device is flexible.

The occlusion device 200 in certain configurations includes a biohazard indicator. In certain configurations this includes a selected color and/or a label. For example, the occlusion device 200 can be the color red. However, it should be appreciated that any color that indicates the materials are biohazardous can be used. For example, the occlusion device 200 can be black, blue, yellow, or orange among other colors. The occlusion device 200 can be multiple colors. The occlusion device 200 can include one face having one color and another face having another color. For example, the occlusion device 200 can include red on the outer face and black on the inner face, thereby indicating which side of the occlusion device should face out. Further the occlusion device 200 can include indicia or a label indicating the container contains biohazardous material. This label can include words “BIOHAZARDOUS” or the universal symbol for biohazardous materials or both. Alternatively, the sterilizing container itself can include a location to include a label. The label can be changeable to indicate whether the sterilizing container is in the sterilization configuration or the transportation configuration.

The occlusion device 200, such as the nonporous covers 200 shown in FIGS. 17A-23 can include a gasket 224. In one configuration, the gasket 224 is permanently affixed to the nonporous cover 200. In another configuration, the gasket 224 is removably affixed to the nonporous cover 200. In yet another configuration, the gasket 224 is not affixed to the nonporous cover 200. It should be appreciated that the gasket 224 can be integral to the nonporous cover 200 or coextensive. The gasket 224 can comprise a material that provides sufficient thickness and integrity to provide a seal with a confronting surface to prevent extraneous materials, including those that are biohazardous, from exiting the container. In one configuration, the gasket 224 is a silicone material. In certain configurations, the gasket 224 is rigid. The gasket 224, in one configuration, is sufficiently rigid to provide structural support to the nonporous cover.

Turning to FIGS. 17A and 17B, provided is an exemplary embodiment of an occlusion device 200 suitable for use in the transportation configuration. Shown in this figure is a nonporous cover 226 and a gasket 224 proximate the perimeter edge of the cover 226. The nonporous cover 226 in one configuration is a sheet of material as described above. The gasket 224 provides a raised portion along the edge of the nonporous cover to provide a sealing interface between two mating surfaces, typically between the sterilizing container 100 and the occlusion device 200 as described above, or between the occlusion device 200 and another mating surface, including, but not limited to a filter door 118 as described above. The gasket 224 material of the occlusion device 200 creates a physical barrier to prevent contaminates from exiting the container 100. In an alternative configuration, the gasket 224 is spaced from the edge of the nonporous cover 226. The nonporous cover 226 includes a length and width that corresponds to the size of the area to be closed. For example, vent ports 124 of FIGS. 3C, 3D, 4E, 4F, and 4G, 13, 14, 15, 16, and access port 116 of FIGS. 5D, 5E, 5F, 5G, 6C, 6D, 6E, 6F, and 12. Additionally, the thickness of the gasket 224 corresponds to a size that is able to fit between the mating surfaces—for example, between the sterilizing container 100 and the occlusion device 200 and/or between the different sections of the filter holder 126. The thickness prevents extraneous materials from exiting sterilizing container 100. The gasket 224 may be separable, permanently affixed, or integral and non-separable from the nonporous cover.

FIGS. 18A and 18B provide an alternative exemplary embodiment of an occlusion device 200 suitable for use in the transportation configuration. Shown in FIG. 18A is a nonporous cover 226 and a gasket 224 at the perimeter edge of the cover 226. In this embodiment, the gasket 224 is formed by folding the edge of the material of the nonporous cover onto itself thereby creating a thicker membrane along the edges of the nonporous cover 226 to prevent contaminates from exiting the container 100. The nonporous cover 226 includes a length and width that corresponds to the size of the area to be closed. For example, vent ports 124 of FIGS. 3C, 3D, 4E, 4F, and 4G, 13, 14, 15, 16, and access port 116 of FIGS. 5D, 5E, 5F, 5G, 6C, 6D, 6E, 6F, and 12. Additionally, the thickness of the gasket 224 corresponds to a size that is able to fit between the mating surfaces—for example, between the sterilizing container 100 and the occlusion device 200 and/or between the different sections of the filter holder 126. The thickness prevents extraneous materials from exiting sterilizing container 100. The gasket 224 may be separable, permanently affixed, or integral and non-separable from the nonporous cover and may be made out of any type of material described above, that is both substantially rigid and able to maintain its integrity during transportation. For example, the gasket 224 may be permanently affixed by use of an adhesive such that the occlusion device 200 will be destroyed if the gasket 224 and nonporous cover 226 are separated. Alternatively, the adhesive used may provide for non-destructive separation of the gasket 224 and the nonporous cover 226.

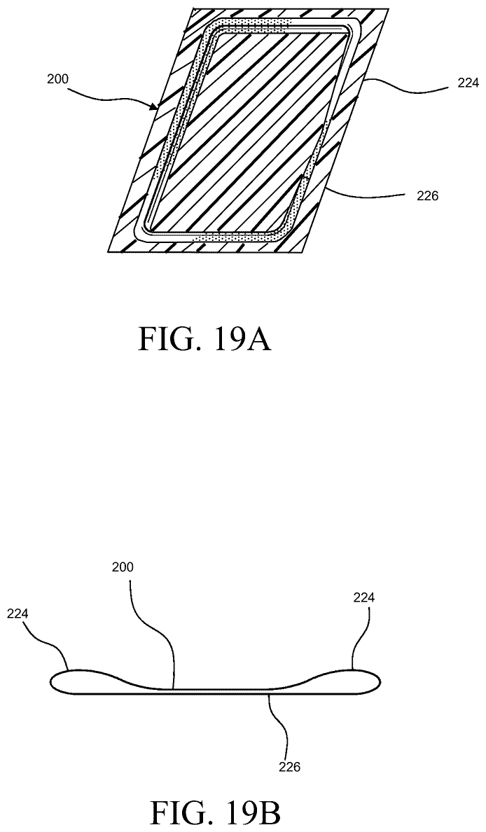

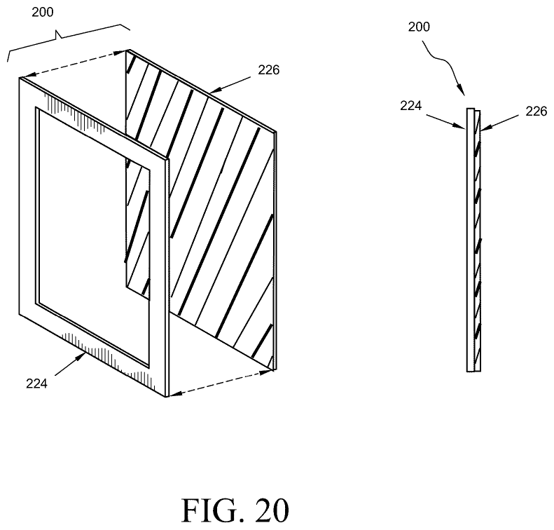

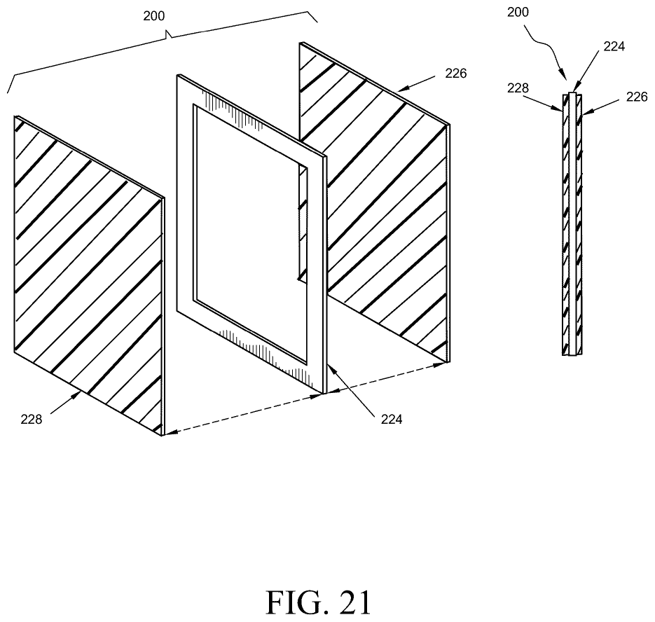

FIGS. 19A and 19B provide another alternative exemplary embodiment of occlusion device 200 suitable for use in the transportation configuration. Shown in FIG. 19A is a nonporous cover 226 and a gasket 224 proximate the perimeter edge of the cover 226. The gasket 224 is comprised of a thicker portion of the nonporous cover 226 material such that gasket 224. Thus, in this configuration, the gasket 224 is integral and not separable from the nonporous cover 226. The nonporous cover 226 includes a length and width that corresponds to the size of the area to be closed. For example, vent ports 124 of FIGS. 3C, 3D, 4E, 4F, and 4G, 13, 14, 15, 16, and access port 116 of FIGS. 5D, 5E, 5F, 5G, 6C, 6D, 6E, 6F, and 12. Additionally, the thickness of the gasket 224 corresponds to a size that is able to fit between the mating surfaces—for example, between the sterilizing container 100 and the occlusion device 200 and/or between the different sections of the filter holder 126. The thickness prevents extraneous materials from exiting sterilizing container 100. The gasket 224 may be made out of any type of material described above, that is both substantially rigid and able to maintain its integrity during transportation. The gasket 224 may be permanently affixed by use of an adhesive such that the occlusion device 200 will be destroyed if the gasket 224 and nonporous cover 226 are separated. Alternatively, the adhesive used may provide for non-destructive separation of the gasket 224 and the nonporous cover 226.

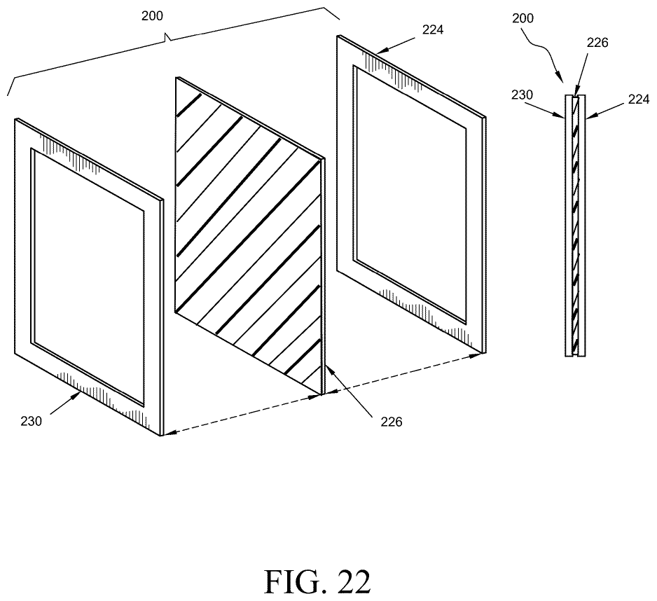

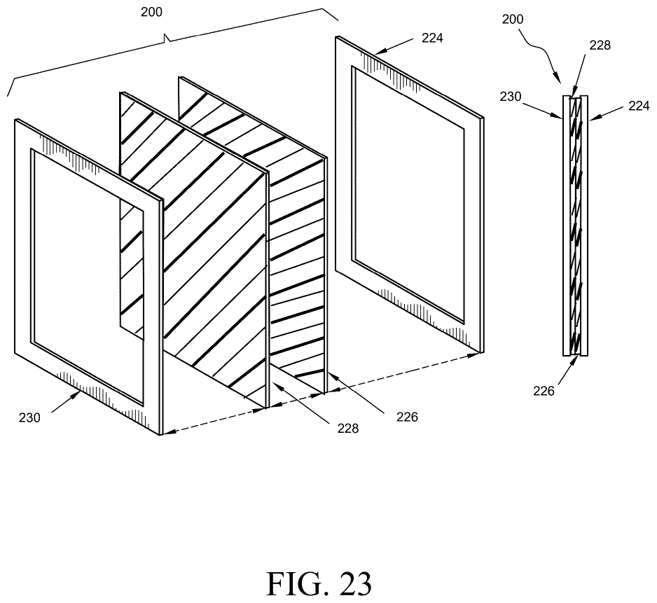

FIGS. 20-23 provide additional exemplary embodiments of occlusion device 200 suitable for use in the transportation configuration. Shown in these figures is a nonporous cover 226 having a gasket 224 around the perimeter edge. As shown in FIG. 21, the occlusion device 200 may include an inner gasket 224 between a first nonporous cover 226 and a second nonporous cover 228. Alternatively, as shown in FIG. 22, the nonporous cover 226 may include a first gasket 224 and a second gasket 230. As shown in FIG. 23, the occlusion device 200 may be two-ply having nonporous cover 226, 228 and first and second gaskets 224, 230 on the outside perimeter edge of each nonporous cover 226, 228. In one configuration, the gasket 224, or gaskets 224, 230 are a substantially rigid frame that is substantially resistant from ripping or tearing. The gaskets 224, 230 may be made out of any type of material described above, that is both substantially rigid and able to maintain its integrity during transportation. For example, the gaskets 224, 230 may be permanently affixed by use of an adhesive such that the occlusion device 200 will be destroyed if the gasket 224, 230 and nonporous cover 226 are separated. Alternatively, the adhesive used may provide for non-destructive separation of the gasket 224, 230 and the nonporous cover 226.

The occlusion device 200 is comprised of a material that is used in place of the porous filters on sterilizing containers 100 to seal the container to prevent leaks and other contamination from exiting the sterilizing container 100. This material closes or occludes, as applicable, the access port 116, vent ports 124, and/or vented port area 125 (for example, openings, fenestrated areas, or holes) making the sterilizing container safe for the transport of contaminated instruments and other objects. The occlusion device 200 can have multiple components, or be a single sheet of material.

In the transportation configuration, the container 100 is sealed to provide a leak-resistant transport system that contains contamination within the container 100. In addition to being leak-resistant, the occlusion device 200 is tear and puncture resistant, thereby reducing the risk of injury and further containing the contaminated object(s) 190. In an exemplary embodiment, the occlusion device 200 is single-use and disposable after one use. In another exemplary embodiment, the occlusion device 200 is re-usable and washable along with the contaminated instruments. In certain exemplary embodiments, the occlusion device 200 is sterilizable. In certain configurations, the occlusion device is a nonporous cover that does not require a gasket. For example, the gasket can be part of the sterilizing container 100. Alternatively, the occlusion device can be made of a material that is capable of forming a seal with a door and sterilizing container. For example, the occlusion device can comprise a solid material such as silicone or a closed-cell elastomeric material that does not require a gasket to form a seal.

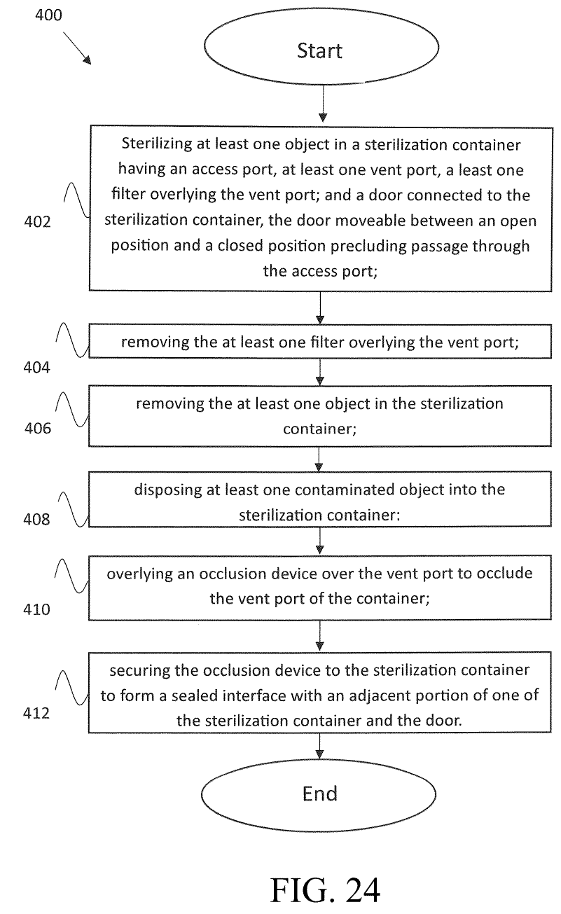

FIG. 24 presents a summary of the above teachings for using a sterilizing container as a transportation container for contaminated objects in the form of a logic diagram. The logic diagram of FIG. 24 can be considered to illustrate the operation of the method. The method 400 starts with block 402 comprising the step of sterilizing at least one object in a sterilizing container having an access port, at least one vent port, a least one filter overlying the vent port; and a door connected to the sterilizing container, the door moveable between an open position and a closed position precluding passage through the access port. Then, according to block 404, the at least one filter overlying the vent port is removed. Block 406 provides that at least one object in the sterilizing container is removed. Then at least one contaminated object is disposed into the sterilizing container according to block 408. The next step, according to block 410 is overlying an occlusion device over the vent port to occlude the vent port of the container. According to block 412, the occlusion device is secured to the sterilizing container to form a sealed interface with an adjacent portion of one of the sterilizing container and the door. In certain configurations, the method 400 further comprises the step of securing the door to the sterilizing container. For example, in the configuration shown in FIG. 20, opening the door permits access to the access port and does not include a vent port. In the configuration shown in FIG. 21, the lid can be secured to the base, and the occlusion device can be placed over the vent port of the lid. Then, the screen door 118 can optionally be secured to the lid or the occlusion device 200 secured to the lid without the screen door 118.

In certain configurations, the step of overlying the occlusion device over the vent port of method 400 includes the step of placing the occlusion device over the vent port on the door and securing the occlusion device and the door to the sterilizing container to occlude the access port and the vent port. For example, in the configuration shown in FIGS. 5A, 12, 14-16 , the occlusion device can be placed over the vent port on the door and then the door having the occlusion device is secured to the sterilizing container. Certain configurations include that the step of securing the occlusion device to the sterilizing container includes the step of engaging at least one clamp on the sterilizing container to form a sealed interface between the occlusion device and the sterilizing container. It should be appreciated that this method streamlines the sterilization process as fewer containers are required for the sterilization process and transportation process.

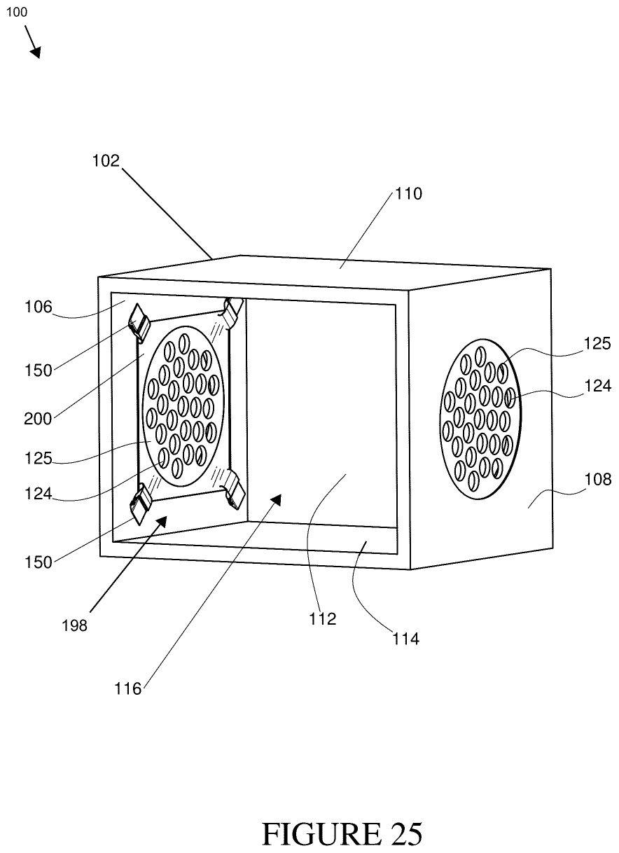

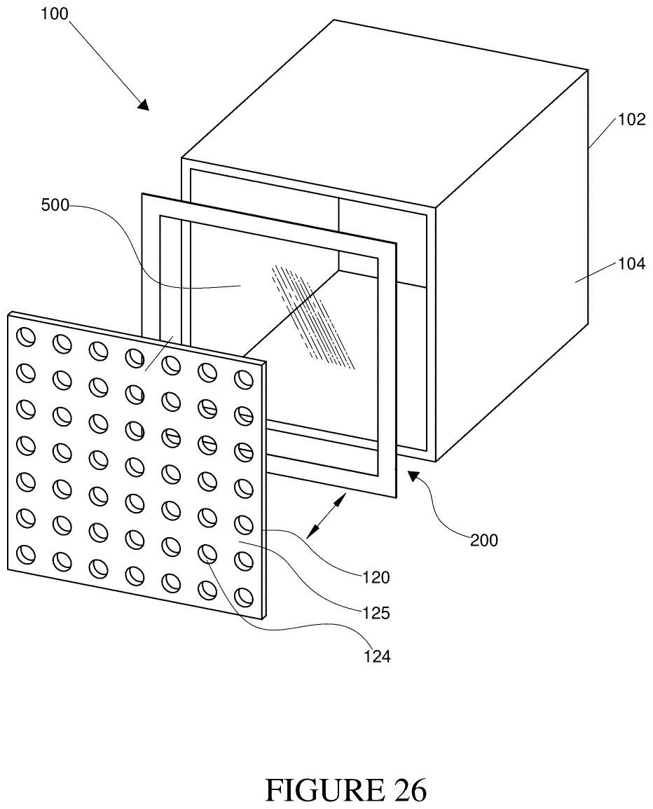

In a further exemplary embodiment, the occlusion device 200 is a nonporous sheet or cover sized to encompass and occlude the vent port of the sterilizing container from an interior 198 of the container 100. For example, in one transportation configuration, the occlusion device 200 overlies and encompasses the vent port(s) on the interior of the door in lieu of the filter according to FIG. 7. In another transportation configuration, the occlusion device 200 overlies and encompasses the vent ports 124 on the inner side(s) of the sterilizing container 100, for example, as shown in FIG. 25. In this configuration, the sterilizing container 100 is a cabinet 102 have an enclosing wall 104 including sidewalls 106, 108, top wall 110, rear wall 112 and bottom wall 114. An access port 116 can be lying in a plane and spaced from the rear wall 112. The sterilizing container 100 further includes a door (not shown) connected to the container 100. The vent area 125 having vent ports 124 in the configuration shown in FIG. 25 are located on the sidewalls 106, 108, however, the vent ports 124 may additionally or alternatively be located on the top wall 110, rear wall 112 and/or bottom wall 114. The sterilizing container 100 can also or alternatively include vent ports 124 on the door. In the transportation configuration, an occlusion device 200 overlies the vent area 125 whether on the enclosing wall 104 or door. The nonporous sheet or cover 200 in one configuration forms a sealed interface with an interior adjacent portion of the at least one of the container 100 and the door 120 as shown in FIG. 26. Thus, when the door precluding access to the access port 116 is closed and the occlusion device 200 is sealed to the cabinet 102, a leak-resistant transport system is provided that contains any contamination within the cabinet 102. In addition to being leak-resistant, the occlusion device 200 is tear and puncture resistant, thereby reducing the risk of injury and further containing the contaminated object(s) 190. In an exemplary embodiment, the occlusion device 200 is single-use and disposable after one use. In another exemplary embodiment, the occlusion device 200 is re-usable and washable along with the contaminated instruments. In certain exemplary embodiments, the occlusion device 200 is sterilizable. In a configuration, the occlusion device 200 includes a seal or gasket 224 (for example as shown in FIGS. 17A-23) for forming a seal 224. The seal 224 in a configuration comprises an edge portion 222 having a thickness sufficient to form a sealed interface between at least one of the sterilizing cabinet 102 and the occlusion device 200 to prevent extraneous materials from exiting the container. In one configuration, the seal 224 is a gasket that is permanently affixed or integral with the occlusion device. In another configuration, the seal 224 is a gasket that is separable from the nonporous cover. In another configuration, the seal 224 is a gasket that is coextensive with the nonporous cover. This seal 224 keeps extraneous materials from exiting the sterilizing cabinet 102 through vents 124, which are covered by the occlusion device 200 and/or 202. In one exemplary embodiment, the seal 224 is a silicone seal. Exemplary embodiments and features of the occlusion device 200, 202 having seal 224 are further described below with respect to FIG. 17A-FIG. 23 which features and configurations are intended to be included in the above-described exemplary embodiments. In certain configurations, the cover has a thickness ranging from approximately 0.5 mils to approximately 3 inches, and more preferably between 3 mil to approximately 1 inch, and more particularly from approximately 5 mil to 40 mil.

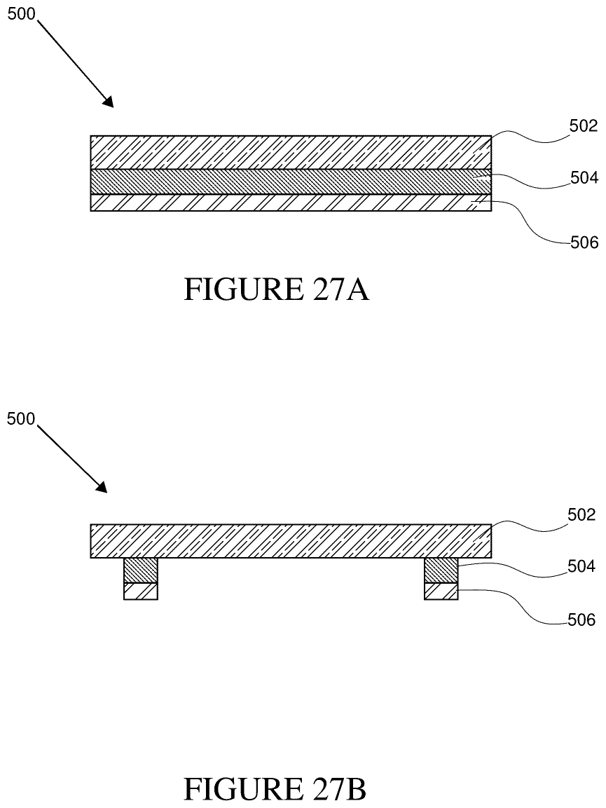

In certain configurations, the occlusion device 200 as provided in each exemplary embodiment is a sheet comprising a nonporous material as shown in FIGS. 27A and 27B. In one configuration, the sheet 500 is a multi-layer film having a first layer 502 and a second layer 504 opposite the first layer 502, the second layer 504 comprising an adhesive for contacting either the surface of the interior of the sterilizing container 100 surrounding the vent port(s) 124 as shown in FIG. 25 or a surface of the door 120 surrounding the vent port(s) 124 to form a seal and preclude contaminants from exiting the container 102. Alternatively, as shown in FIG. 26, the sheet 500 forms a seal with the perimeter of the enclosing walls 104 to preclude contaminants from existing the container 102.

The sheet 500, as shown in FIGS. 27A and 27B, in certain configurations is a flexible material. The adhesive may be pressure-sensitive such that a seal forms when pressure is applied to the sheet 500. The first layer 502 in certain configurations has a thickness ranging from approximately 1.5 mils to approximately 4.0 mils. In one embodiment, the thickness is approximately 2.0 mils to 3.0 mils. The first layer 502 in certain configurations is comprised of polyethylene. For example the first layer may comprise high-density polyethylene (HDPE) or low-density polyethylene (LDPE). However, other types of materials may be used, including but not limited to plastics and cornstarch-based polymers. Preferably, the sheet 500 exceeds the Dart Impact Resistance Test Requirement of 165 grams according to ASTM D1709 and meets or exceeds the propagation tear resistance standard according to ASTM D1922. As shown in FIG. 27A, the sheet 500 in certain configurations may include a third layer 506 which is a protective outer layer overlying the adhesive layer 504. The protective outer layer 506 may be peeled from the second layer 504 before application to expose the adhesive. In certain configurations, as shown in FIG. 27A, the adhesive layer 504 circumscribes the first layer 502 proximate a perimeter edge of the first layer in a frame shape having a hollow center with a strip of adhesive approximately ¼″ to 2 inches in width. The adhesive 504 is configured to contact the surface of the interior of the sterilizing container 102 or a surface of the door 120 surrounding the vent port(s) 124 to form a seal and preclude contaminates from existing the container.

In certain configurations, the sheet 500 is disposable after a single-use. In certain configurations, the sheet 500 is autoclavable and can withstand temperatures of up to 250° F. It should be appreciated that the sheet 500 is sized to extend beyond the area of the vent port 124 to fully encompass the vent port 124. Thus, the perimeter of the sheet 500 engages or adheres to the surface area surrounding the vent port 124.

As provided above, the occlusion device 200 in certain configurations includes a biohazard indicator. In certain configurations this includes a selected color and/or a label. For example, the occlusion device 200 can be the color red. However, it should be appreciated that any color that indicates the materials are biohazardous can be used. For example, the occlusion device 200 can be black, blue, yellow, or orange among other colors. The occlusion device 200 can be multiple colors. The occlusion device 200 can include one face having one color and another face having another color. For example, the occlusion device 200 can include red on the outer face and black on the inner face, thereby indicating which side of the occlusion device should face out. Further the occlusion device 200 can include indicia or a label indicating the container contains biohazardous material. This label can include words “BIOHAZARDOUS” or the universal symbol for biohazardous materials or both. Alternatively, the sterilizing container itself can include a location to include a label. The label can be changeable to indicate whether the sterilizing container is in the sterilization configuration or the transportation configuration.

The occlusion device 200 in certain configurations includes a gasket 224 as shown in FIGS. 17A-23 The gasket 224 includes an edge portion with a thickness sufficient to form a sealed interface between at least one of the sterilizing container and the door to prevent extraneous materials from exiting the container. The gasket 224 in certain configurations is permanently affixed or integral with the nonporous cover. Alternatively the gasket 224 is separable from the nonporous cover. In certain configurations, the gasket is coextensive with the nonporous cover.

As shown in FIG. 25, the container 100 may include a securing mechanism such as at least one tab, clamp, latch, hinge, hanger, or hook 150 to secure the occlusion device 200 to the interior of the sterilizing container 100 such that the occlusion device 200 overlies the vent port 124. Alternatively, the door 120 having the vent port 124 can include at least one tab, clamp, latch, hinge, or hook 168 to secure the occlusion device 200 to the door 120 as shown in FIG. 9.

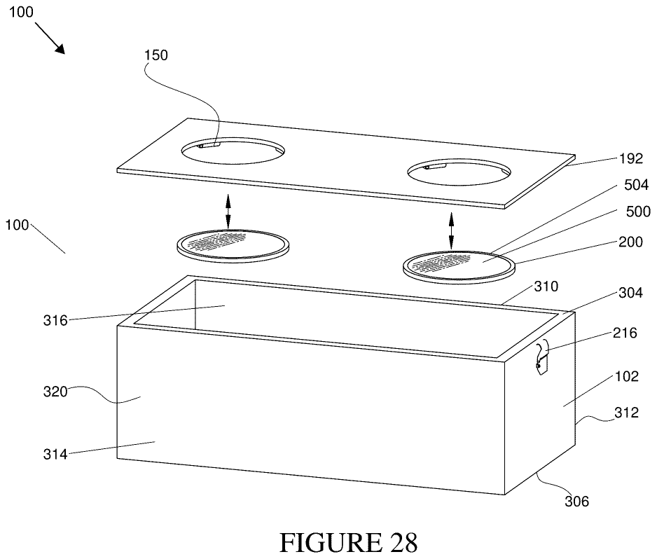

As shown in FIG. 28, it should be appreciated that the container 100 having base features as shown and described above with respect to and in FIGS. 14, 15 and 16 may include an occlusion device 200 that seals to the inner portion of lid 192. For example, sheet 500, as shown and described above with respect to and in FIGS. 27A and 27B may be removably adhered or removably secured to the inside of lid 192 to encompass each vent port 124. In one configuration, one occlusion device 200 covers one vent port 124. In another configuration, one occlusion device 200 covers all the vent ports 124. As provided above, it should be appreciated that the sheet 500 is sized to extend beyond the area of the vent port 124 such that the sheet 500 fully encompasses the vent port 124. Thus, the perimeter of the sheet 500 engages or adheres to the surface area surrounding the vent port 124. Alternatively, the occlusion device 200 is a cover having a thickness and rigidity for occluding the vent ports 124. By rigidity it is meant that the occlusion device is capable of holding its shape without further support. The occlusion device 200 in one configuration forms a sealed interface with an adjacent portion of the lid 192 such that during the transportation of the container 100 having at least one object 190, which can include at least one tray 160 (for example as shown in FIG. 12), the container 100 is sealed to provide a leak-resistant transport system that contains contamination within the container 100. In addition to being leak-resistant, the occlusion device 200 is tear and puncture resistant, thereby reducing the risk of injury and further containing the contaminated object(s) 190. In an exemplary embodiment, the occlusion device 200 is single-use and disposable after one use. In another exemplary embodiment, the occlusion device 200 is re-usable and washable along with the contaminated instruments. In certain exemplary embodiments, the occlusion device 200 is sterilizable. The container 100 may include a securing mechanism such as at least one tab, clamp, latch, hinge, hanger, or hook 150 to secure the occlusion device 200 to the interior of the lid 192 such that the occlusion device 200 overlies the vent port 124.

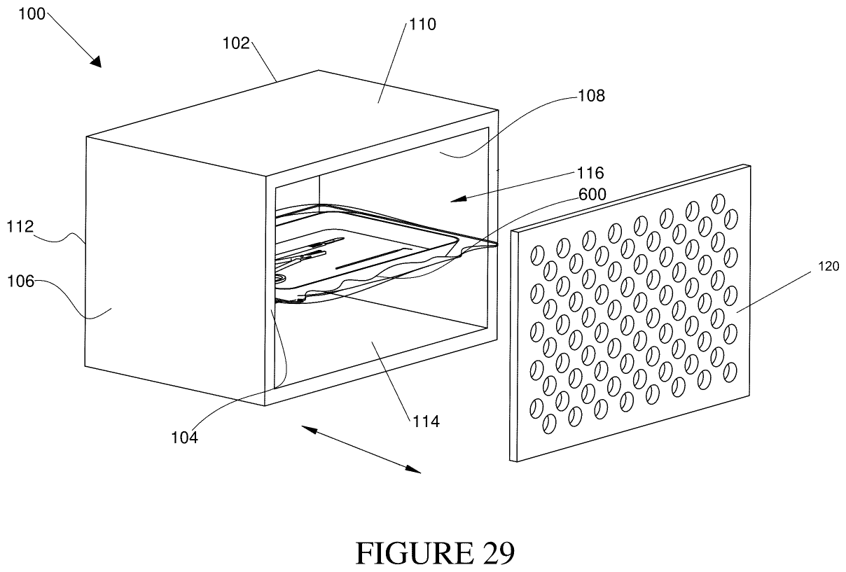

Turning now to FIG. 29, shown is a contaminated object 190 encased in an occlusion device 200. In this embodiment, the occlusion device 200 is a nonporous material comprising a flexible film that is resistant to punctures or tears to preclude contaminants from exiting the container. For example, in one configuration, the occlusion device 200 is a bag 600 that meets or exceeds the Dart Impact Resistance Test Requirement of 165 grams according to ASTM D1709 and that meets or exceeds the propagation tear resistance standard according to ASTM D1922. The bag 600 includes a thickness ranging from approximately 1.5 mils to approximately 4.0 mils. In one embodiment, the thickness is approximately 2.0 mils to 3.0 mils. The bag 600 may include an opening that can be securely fastened. For example, the bag 600 may include a zipper-lock closure, an adhesive closure or a zip-tie closer. The bag 600 in certain configurations is a flexible material. In certain configurations, the bag comprises high-density polyethylene (HDPE) or low-density polyethylene (LDPE). However, other types of materials may be used, including but not limited to plastics and cornstarch-based polymers. In certain configurations, the bag 600 is disposable after a single-use. In certain configurations, the bag 600 is autoclavable and can withstand temperatures of up to 250° F. It should be appreciated that the bag 600 is sized to fully encompass contaminated object 190, which may include a tray having contaminated objects.

As provided above, the occlusion device 200 in certain configurations includes a biohazard indicator. In certain configurations this includes a selected color and/or a label. For example, the occlusion device 200 can be the color red. However, it should be appreciated that any color that indicates the materials are biohazardous can be used. For example, the occlusion device 200 can be black, blue, yellow, or orange among other colors. The occlusion device 200 can be multiple colors. The occlusion device 200 can include one face having one color and another face having another color. For example, the occlusion device 200 can include red on the outer face and black on the inner face, thereby indicating which side of the occlusion device should face out. Further the occlusion device 200 can include indicia or a label indicating the container contains biohazardous material. This label can include words “BIOHAZARDOUS” or the universal symbol for biohazardous materials or both. Alternatively, the sterilizing container itself can include a location to include a label. The label can be changeable to indicate whether the sterilizing container is in the sterilization configuration or the transportation configuration.

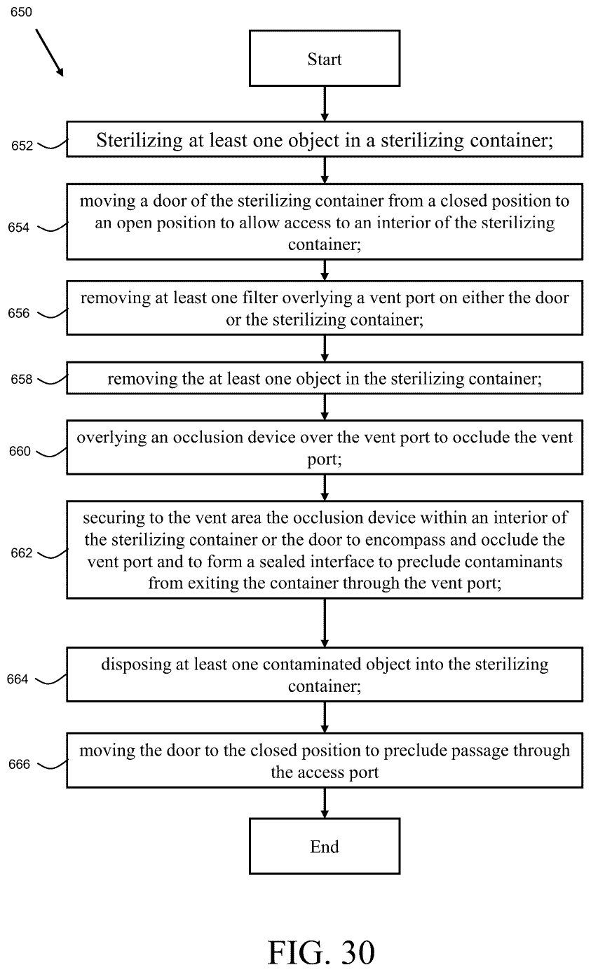

FIG. 30 presents a summary of the above teachings for using a sterilizing container as a transportation container for contaminated objects by securing an occlusion device 200 to the interior of the sterilizing container or door in the form of a logic diagram. The logic diagram of FIG. 30 can be considered to illustrate the operation of the method. The method 650 starts with block 652 comprising the step of sterilizing at least one object in a sterilizing container 100. The sterilizing container 100 includes an access port 116 to allow access to an interior of the sterilizing container, a door connected to the sterilizing container 100, the door moveable between an open position and a closed position precluding passage through the access port 116, at least one vent area 125 on the door or the sterilizing container 100, the vent area 125 having at least one vent port 124, and at least one filter overlying the vent port 124. According to step 654, the door is moved from the closed position to the open position to allow access to the interior of the sterilizing container 100 through the access port 116. Next, according to step 656, the at least one filter overlying the vent port 124 is removed. Additionally, according to step 658, the at least one object 190 in the sterilizing container 100 is removed. According to step 660, the occlusion device 200 is laid over the vent port area 125 to occlude the vent port 124. The occlusion device 200 is secured to the vent port area within the interior of the sterilizing container 100 or the door 120 to encompass and occlude the vent port 124 and to form a sealed interface to preclude contaminants from exiting the container through the vent port 124, according to step 662. Next, as provided in step 664, at least one contaminated object 190 is disposed into the sterilizing container 100 and the door 120 is moved to the closed position to preclude passage through the access port according to step 666. It should be appreciated that step 662 may include the step of engaging at least one clamp 150 in the interior of the sterilizing container 100 to form a sealed interface between the occlusion device 200 and the sterilizing container 100, wherein the clamp 150 secures the occlusion device 200 in place. In the configuration where the occlusion device 200 occludes vent ports on the door 120, it should be appreciated that clamps may be located on the door. In certain configurations, the occlusion device 200 comprises a gasket having an edge portion with a thickness sufficient to form a sealed interface between at least one of the container and the door to prevent extraneous materials from exiting the container. It should be appreciated that the term door when used to describe the method shown in FIG. 30 may refer to door 120 or lid 192.

In certain configurations, step 660 of overlying the occlusion device 200 over the vent port 124 includes the step of applying a nonporous sheet 500 comprising a multi-layer film having a first layer 502 and a second layer 504 opposite the first layer 502, the second layer 504 comprising an adhesive for contacting one of the vent area 125 of the interior of the sterilizing container 100 surrounding the vent port 124 or a vent area 125 of the door 120 surrounding the vent port 124 to form a seal and preclude contaminants from exiting the container. Step 660 may further include the step of removing a protective outer layer 506 overlying the second adhesive layer 504 before overlying the occlusion device 200 over the vent area 125. In certain configurations, a pressure is applied to the occlusion device 200 to form a seal between the nonporous sheet 500 and the vent area 125 of the interior of the sterilizing container 100 surrounding the vent port 125 or the vent are 125 of the door 120 surrounding the vent port 124.

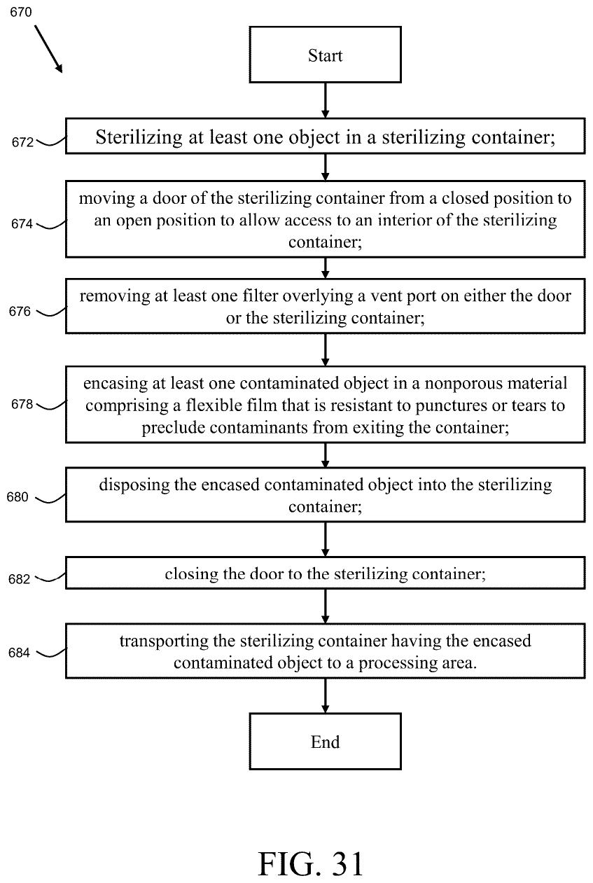

Turning now to FIG. 31 this figure presents a summary of the above teachings for using a sterilizing container as a transportation container for contaminated objects by encasing at least one contaminated object in a nonporous material in the form of a logic diagram. The logic diagram of FIG. 31 can be considered to illustrate the operation of the method. The method 670 starts with block 672 comprising the step of sterilizing at least one object 190 in a sterilizing container 100 having an access port 116 to allow access to an interior of the sterilizing container 100, a door connected to the sterilizing container 100, the door moveable between an open position and a closed position precluding passage through the access port 116, at least one vent area 125 on the door or the sterilizing container 100, the vent area 125 having at least one vent port, and at least one filter overlying the vent port. Next, according to step 674, the door is moved from the closed position to the open position to allow access to the interior of the sterilizing container 100. Step 676 includes removing the at least one filter overlying the vent port on either the door or the sterilizing container. Additionally, step 678 provides removing the at least one object 190 in the sterilizing container 100. Next, according to step 680, the at least one contaminated object 190 is encased in a nonporous material comprising a flexible film that is resistant to punctures or tears to preclude contaminants from exiting the container. Next, the encased contaminated object 190 is disposed into the sterilizing container 100 and the door to the sterilizing container is closed according to step 682. The sterilizing container 100 having the encased contaminated object 190 can then be transported to a processing area according to step 684. In certain configurations, step 680 includes the step of placing the object in a bag that meets or exceeds the Dart Impact Resistance Test Requirement of 165 grams according to ASTM D1709 and that meets or exceeds the propagation tear resistance standard according to ASTM D1922. Step 680 may further include the step of securely sealing the bag closed before disposing the encased contaminated object into the sterilizing container. It should be appreciated that the term door when used to describe the method shown in FIG. 31 may refer to door 120 or lid 192. For clarity, it should be understood that the vent area 125 of each configuration described herein may include a single vent port or a plurality of vent ports and the term vent port 124 is intended to include either a single vent port configuration or a plurality of vent ports as applicable.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. An occlusion device for a vent port of a sterilizing container, the sterilizing container having an access port, a door connected to the sterilizing container, the door moveable between an open position permitting passage through the access port to an interior of the sterilizing container and a closed position precluding passage through the access port, at least one of the sterilizing container and the door having a vent port, the occlusion device comprising:
a nonporous sheet sized to encompass and occlude the vent port of the sterilizing container, the nonporous sheet comprising a material that is resistant to punctures or tears.

2. The occlusion device of claim 1 wherein the nonporous sheet comprises a multi-layer film having a first layer and a second layer opposite the first layer, the second layer comprising an adhesive for contacting one of a surface of the interior of the sterilizing container surrounding the vent port or a surface of the door surrounding the vent port to form a seal and preclude contaminants from exiting the container.

3. The occlusion device of claim 2, wherein the adhesive circumscribes the first layer proximate a perimeter edge of the first layer, wherein the adhesive is configured to contact the surface of the interior of the sterilizing container or a surface of the door surrounding the vent port to form a seal and preclude contaminates from existing the container.

4. The occlusion device of claim 2, wherein the nonporous sheet comprises a flexible material and wherein the adhesive is a pressure-sensitive.

5. The occlusion device of claim 1, wherein the nonporous sheet meets or exceeds the dart impact resistance test meets or exceeds the Dart Impact Resistance Test Requirement of 165 grams according to ASTM D1709.

6. The occlusion device of claim 1, wherein the nonporous sheet further comprises a gasket having an edge portion with a thickness sufficient to form a sealed interface between at least one of the sterilizing container and the door to prevent extraneous materials from exiting the container.

7. The occlusion device of claim 1, wherein the nonporous sheet comprises a material selected from the group consisting of: high-density polyethylene (HDPE), polypropylene plastic, plastic, polyethylene, and cornstarch-based polymers.

8. The occlusion device of claim 1, wherein the occlusion device is clamped to the interior of one of the sterilizing container having the vent port and the door having the vent port.

9. The occlusion device of claim 1, wherein the occlusion device is disposable and further comprises a biohazard material indicator.

10. The occlusion device of claim 1, wherein the biohazard material indicator includes at least one of a biohazard symbol and a selected color.

11. A method for using a sterilizing container as a transportation container for contaminated objects, the method comprising:
(a) sterilizing at least one object in a sterilizing container having an access port to allow access to an interior of the sterilizing container, a door connected to the sterilizing container, the door moveable between an open position and a closed position precluding passage through the access port, at least one vent area on the door or the sterilizing container, the vent area having at least one vent port, and at least one filter overlying the vent port;
(b) moving the door from the closed position to the open position to allow access to the interior of the sterilizing container;
(c) removing the at least one filter overlying the vent port;
(d) removing the at least one object in the sterilizing container;
(e) overlying an occlusion device over the vent port area to occlude the vent port;
(f) securing to the vent area the occlusion device within an interior of the sterilizing container or the door to encompass and occlude the vent port and to form a sealed interface to preclude contaminants from exiting the container through the vent port;
(g) disposing at least one contaminated object into the sterilizing container; and
(e) moving the door to the closed position to preclude passage through the access port.

12. The method according to claim 11, wherein in the step of securing the occlusion device comprises the step of engaging at least one clamp in the interior of the sterilizing container to form a sealed interface between the occlusion device and the sterilizing container.

13. The method according to claim 11, wherein the step of securing the occlusion device comprises the step of engaging at least one clamp on the door to form a sealed interface between the occlusion device and the door.

14. The method according to claim 11, wherein the occlusion device comprises a gasket having an edge portion with a thickness sufficient to form a sealed interface between at least one of the container and the door to prevent extraneous materials from exiting the container.

15. The method according to claim 11, wherein the step of overlying an occlusion device over the vent port includes the step of applying a nonporous sheet comprising a multi-layer film having a first layer and a second layer opposite the first layer, the second layer comprising an adhesive for contacting one of the vent surface of the interior of the sterilizing container surrounding the vent port or a vent surface of the door surrounding the vent port to form a seal and preclude contaminants from exiting the container.

16. The method according to claim 15, further comprising removing a protective outer layer overlying the second adhesive layer before overlying the occlusion device over the vent port.

17. The method according to claim 15, further comprising the step of applying pressure to the occlusion device to form a seal between the nonporous sheet and the vent surface of the interior of the sterilizing container surrounding the vent port or the vent surface of the door surrounding the vent port.

18. A method for using a sterilizing container as a transportation container for contaminated objects, the method comprising:
sterilizing at least one object in a sterilizing container having an access port to allow access to an interior of the sterilizing container, a door connected to the sterilizing container, the door moveable between an open position and a closed position precluding passage through the access port, at least one vent area on the door or the sterilizing container, the vent area having at least one vent port, and at least one filter overlying the vent port;
(a) sterilizing at least one object in a sterilizing container having an access port to allow access to an interior of the sterilizing container, a door connected to the sterilizing container, the door moveable between an open position and a closed position precluding passage through the access port, at least one vent area on the door or the sterilizing container, the vent area having at least one vent port, and at least one filter overlying the vent port;
(b) moving the door from the closed position to the open position to allow access to the interior of the sterilizing container;
(c) removing the at least one filter overlying the vent port on either the door or the sterilizing container;
(c) removing the at least one object in the sterilizing container;
(c) encasing at least one contaminated object in a nonporous material comprising a flexible film that is resistant to punctures or tears to preclude contaminants from exiting the container;
(d) disposing the encased contaminated object into the sterilizing container;
(e) closing the door to the sterilizing container; and
(f) transporting the sterilizing container having the encased contaminated object to a processing area.

19. The method of claim 18, wherein the step of encasing the at least one contaminated object in a nonporous material includes the step of placing the object in a bag that meets or exceeds the Dart Impact Resistance Test Requirement of 165 grams according to ASTM D1709 and that meets or exceeds the propagation tear resistance standard according to ASTM D1922.

20. The method of claim 18, wherein the step of encasing the at least one contaminated object in a nonporous material includes the step of securely sealing the bag closed before disposing the encased contaminated object into the sterilizing container.
